(19) United States
(12) Patent Application Publication
(10) Pub. No.: US 2026/0217920 A1
(43) Pub. Date: Jul. 30, 2026

(54) Composite Nanoparticles, Compositions thereof, and Methods of Use and Manufacture

(71) Applicant: Van Andel Research Institute, Grand Rapids, MI (US)

(72) Inventor: Michael Patrick Vincent, Grand Rapids, MI (US)

(21) Appl. No.: 19/456,728

(22) Filed: Jan. 22, 2026

Related U.S. Application Data
(60) Provisional application No. 63/749,256, filed on Jan. 24, 2025.

(51) Int. Cl. C08G 81/02; A61K 9/107; A61K 9/51; C08K 5/101; C08K 5/3415

(52) U.S. Cl. CPC C08G 81/027; A61K 9/1075; A61K 9/5123; A61K 9/5153; C08K 5/101; C08K 5/3415

(57) ABSTRACT

A composite nanoparticle that includes a biodegradable amphiphilic material and a hydrophobic compound having a stable isotope label. The biodegradable amphiphilic material may be a polymeric, non-polymeric, or hybrid polymer/lipid self-assembling system. The composite nanoparticle may be a poly(ethylene glycol)-block-poly(ε-caprolactone) (PEG-b-PCL) copolymer, a poly(ethylene glycol)-block-poly(L-lactide) (PEG-b-PLA) copolymer, or poly(ethylene glycol)-block-poly(lactide-co-glycolide) (PEG-b-PLGA) copolymer; and the poly(ethylene glycol) block of any of the polymeric biodegradable amphiphilic copolymers may be poly(ethylene glycol) or methoxy poly(ethylene glycol). The hydrophobic compound of the composite nanoparticle may be a fatty acid, a fatty acid derivative, and a lipid analog having a stable isotope label. Compositions and methods of using and making the composite nanoparticle.

### CROSS-REFERENCE TO RELATED APPLICATIONS

This application claims priority to U.S. Provisional Application 63/749,256 filed on Jan. 24, 2025, the disclosure of which is considered part of the disclosure of this application and is hereby incorporated by reference in its entirety.

### TECHNICAL FIELD

This invention relates to composite nanoparticles, compositions thereof, and methods of using and making composite nanoparticles and compositions of composite nanoparticles.

### BACKGROUND OF INVENTION

Stable isotope labeling (SIL) is a technique that allows for the movement, incorporation, and metabolism of molecules to be tracked within biological or chemical systems without altering their chemical behavior. This technique relies on the use of molecular tracers that incorporate non-radioactive isotopes that differ in the number of neutrons compared to the most abundant form (e.g., carbon-13 (¹³C or 13C), hydrogen-2 (2H or 2H; deuterium (D)), nitrogen-15 (¹⁵N), oxygen-18 (¹⁸O), etc.). Common examples of these molecular tracers include uniformly labeled ¹³C glucose (U-¹³C-glucose) or uniformly labeled palmitate (U-¹³C-palmitate), among others, which incorporate the specified isotope (¹³C in the provided examples) in place of the most abundant form of the element (¹²C in the provided examples) in the molecule. Many variations of such tracers exist, including those that are not fully labeled with the specified isotopes and only partially incorporate the specified isotopes. Metabolomics, lipidomics, and proteomics investigations employing mass spectrometry (MS), nuclear magnetic resonance (NMR), or other suitable techniques follow the incorporation of these isotopes into other biomolecules as the tracer undergoes chemical and/or biochemical transformations through its participation in metabolic processes.

Hydrophilic tracers such as U-¹³C-glucose are easy to use in research or clinical applications due to their high solubility in aqueous media. In contrast, the use of hydrophobic tracers in these applications remains severely limited by several practical challenges. This is a key issue that limits the scope of the information that can be obtained from the aforementioned applications.

Hydrophobic tracers, such as isotopically labeled lipids, provide fundamental insights into lipid metabolism (fatty acid uptake and oxidation, lipid biosynthesis and remodeling, lipid transport, etc.) and related disease mechanisms (including the diagnosis of lipid metabolism dysregulation in cancer, metabolic disorders, cardiovascular diseases, neurodegenerative diseases, etc.).

Hydrophobic stable isotope tracers that are available for probing cellular metabolism have properties that limit their use in research and clinical applications, including in vitro, ex vivo, and in vivo studies. These challenging properties include low solubility in water, biofluids, or other aqueous media, as well as issues with poor bioavailability, instability due to degradation or other mechanisms, poor uptake/internalization by cell types of interest, rapid clearance from the immune system, and issues with acute toxicity and immunogenicity. These properties limit the bioavailability of the hydrophobic tracer and its ability to participate in metabolic reactions, and can produce adverse effects (toxicity, etc.) that confound interpretation of the metabolism of the tracer.

Hydrophobic tracers incorporating stable isotope labels, such as carbon-13 (¹³C) are of particular interest for use in probing lipid metabolism. Prominent examples of these tracers are 13C-labeled fatty acids (FAs), such as palmitate (FA 16:0) that is uniformly labeled with carbon-13 (U-¹³C-palmitate). Tracing palmitate metabolism is of great interest due to its status as the most abundant dietary saturated fatty acid, but it is challenging to use as it is insoluble in aqueous media due to its high hydrophobicity (log P 6.4; see PubChem CID 985 for chemical properties).

Currently, U-¹³C-palmitate is typically prepared as a non-covalent complex with bovine serum albumin (BSA). The BSA-mediated delivery of palmitate suffers from a variety of procedural and biological issues. These include the use of denaturing conditions, such as the heat and organic solvent exposure, as well as issues with toxicity and immunogenicity. For example, it is known that fatty acid-free BSA, including product variants marketed as being endotoxin-free, are commonly contaminated with lipopolysaccharides (LPS) and lipopeptides that produce pro-inflammatory responses mediated by toll-like receptor 2 (TLR2) and TLR4. These issues confound the interpretation of metabolism studies, but they also limit the use of these fatty acid tracers in vivo.

There is a need for a delivery system that overcomes the aforementioned issues with tracer delivery. In particular, there is a need for nanocarrier platforms that encapsulate hydrophobic tracers and/or metabolism-altering compounds to enable their effective transport to tissues and cells without the production of confounding or adverse effects.

### SUMMARY OF THE INVENTION

One embodiment of the invention is a composite nanoparticle that includes (a) a biodegradable amphiphilic material and (b) a hydrophobic compound having a stable isotope label. In one aspect, the biodegradable amphiphilic material is a polymeric, non-polymeric, or hybrid polymer/lipid self-assembling system. In further aspects of the invention, the biodegradable amphiphilic material may be polymeric and any of poly(ethylene glycol)-block-poly(ε-caprolactone) (PEG-b-PCL), poly(ethylene glycol)-block-poly(L-lactide) (PEG-b-PLA), or poly(ethylene glycol)-block-poly(lactide-co-glycolide) (PEG-b-PLGA).

In further embodiments, the amphiphilic material may include other self-assembly polymers, copolymers, or polymer blends, including but not limited to poly(ethylene glycol)-block-polycarbonate, poly(ethylene glycol)-block-polyester, poly(ethylene glycol)-block-poly(amino acid), poly(ethylene glycol)-block-poly(beta-amino ester), poly(ethylene glycol)-block-poly(ortho ester), or combinations thereof. In other embodiments, the amphiphilic material comprises non-polymeric or hybrid self-assembling systems such as phospholipids, ionizable lipids, cationic lipids, neutral lipids, cholesterol, or mixtures thereof, which may form micelles, liposomes, lipid nanoparticles (LNPs), or polymersomes. In certain aspects, the composite nanoparticle may be a hybrid structure comprising both polymeric and lipid components. Further, the biodegradable amphiphilic material may be a tri-block copolymer, for example, a poly(ethylene glycol)-block-poly(lactide)-block-poly(ethylene glycol) copolymer (PEG-b-PLA-b-PEG).

In some embodiments, the amphiphilic material includes a hydrophilic segment and a hydrophobic segment. The hydrophilic segment of the amphiphilic copolymer may comprise poly(ethylene glycol) or poly(ethylene glycol) derivatives terminated with functional groups including, but not limited to, a hydroxyl group (—OH), a methyl group (—CH₃), a phosphate group (—PO₄), an amine group (—NH₂), a carboxyl group (—CO₂H), a thiol group (—SH), an aldehyde group (—CHO), a vinyl group (—C₂H₃), an epoxide group, a malemide group, or an azide group (N₃); or the poly(ethylene glycol) block of any of the biodegradable amphiphilic materials may be methoxy poly(ethylene glycol). The hydrophobic segment may contain aliphatic polyesters, polycarbonates, polyethers, polyurethanes, or lipid tails. In a further embodiment, the hydrophobic segment is poly(lactide) which may be poly(L-lactide) or poly(D,L-lactide). In further embodiments, the hydrophobic segment may be poly(ε-caprolactone), poly(lactide), or poly(lactide-co-glycolide); the hydrophobic segment may be poly(lactide) in the form of poly(L-lactide), poly(D-lactide), poly(D,L-lactide), or combinations thereof, or the hydrophobic segment may be poly(lactide-co-glycolide) and the lactide component is in the form of poly(L-lactide), poly(D-lactide), poly(D,L-lactide), or combinations thereof. In a further embodiment, the hydrophobic segment is poly(lactide-co-glycolide), wherein the lactide and glycolide units may be present in any molar ratio, and wherein the lactide component may comprise poly(L-lactide), poly(D-lactide), poly(D,L-lactide), or combinations thereof.

The hydrophobic compound of the composite nanoparticle may include fatty acids, fatty acid derivatives, or lipid analogs. In some embodiments, the hydrophobic compound may be palmitate, palmitic acid, oleate, oleic acid, palmitoleate, stearate, linoleate, linolenate, arachidonate, glycerol, triolein, tripalmitin, triglyceride, cholesterol, acetate, choline, ethanolamine, mevalonate, isoprenoids, ceramides, sphingolipids, or other hydrophobic tracers or metabolites containing stable isotope labels.

In some embodiments, the poly(ethylene glycol) block of any of the biodegradable amphiphilic copolymers may be poly(ethylene glycol), methoxy poly(ethylene glycol), or other forms of poly(ethylene glycol) terminated with alternative chemical groups (such as a phosphate).

In other embodiments of the invention, the stable isotope label of the hydrophobic compound may be any of carbon-13 (¹³C), hydrogen-2 (2H), nitrogen-15 (¹⁵N), or oxygen-¹⁸ (¹⁸O); or the hydrophobic compound may be U-¹³C-palmitate. In a further embodiment, the composite nanoparticle of the invention also includes a hydrophobic fluorescent dye.

In further aspects of the invention, the composite nanoparticle may have an average diameter that ranges from about 20 nm to about 400 nm; or the average diameter of the plurality of composite nanoparticles may range from about 20 nm to about 50 nm. In another embodiment, the composite nanoparticle may have a surface zeta potential in the range of about −50 mV to about +50 mV; or the surface zeta potential may be in the range of about −20 mV to about −10 mV.

Yet another embodiment of the invention is a composition of a plurality of composite nanoparticles in which each composite nanoparticle of the plurality of composite nanoparticles includes a biodegradable amphiphilic material and a hydrophobic compound, and the hydrophobic compound includes a stable isotope label.

In another embodiment of the invention, the composition is a pharmaceutical composition having (a) a plurality of composite nanoparticles in which each composite nanoparticle of the plurality of composite nanoparticles includes a biodegradable amphiphilic material and a hydrophobic compound, and the hydrophobic compound includes a stable isotope label; and (b) a pharmaceutically acceptable carrier.

Other aspects of the invention include uses of the inventive composite nanoparticles and compositions thereof. In one aspect, the method is following a hydrophobic compound through a biochemical pathway in a cell, including: providing the cell, providing the inventive composite nanoparticle, delivering an effective amount of the composite nanoparticle to the cell, and following the hydrophobic compound through the biochemical pathway in the cell. The biochemical pathway may be central carbon metabolism, lipid biosynthesis, lipid remodeling, or lipid transport, or any associated pathway involving the delivered hydrophobic compound or products formed through its participation in one or more biological reactions.

Another aspect of the invention is a method of tracking internalization of a hydrophobic compound in a cell, including providing the cell, providing the inventive composite nanoparticle, delivering an effective amount of the composite nanoparticle to the cell, and tracking internalization of the hydrophobic compound by the cell.

Yet another aspect of the invention is a method of tracking distribution of a hydrophobic compound in a biological organ or tissue, including providing the biological organ or tissue, providing the inventive composite nanoparticle, delivering an effective amount of the composite nanoparticle to the biological organ or tissue and tracking distribution of the hydrophobic compound in the biological organ or tissue.

A further aspect of the invention is method of diagnosing a condition or a disease in a subject, including providing the inventive composition, administering a diagnostically effective amount of the composition to the subject or a cell or tissue of the subject, and tracking the movement, incorporation, or metabolic effect of the composite nanoparticle in the subject, cell, or tissue.

Other aspects of the invention are methods of making a composite nanoparticle, where the composite nanoparticle includes a biodegradable amphiphilic material and a hydrophobic compound having a stable isotope label, and where the method steps include providing the biodegradable amphiphilic material and the hydrophobic compound, and loading the hydrophobic compound into the biodegradable amphiphilic material either during or after self-assembly of the biodegradable amphiphilic material.

The foregoing and other aspects and advantages of the invention will appear from the following description. In the description, reference is made to the accompanying drawings which form a part of hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIGS. 1A-1E show the physicochemical characterization of nanocarrier formulations developed for metabolite and lipid tracing applications. FIG. 1A is an illustration of nanocarrier types used in this study. These systems are polymeric micelles self-assembled from biodegradable linear block copolymer amphiphiles consisting of a methoxy poly(ethylene glycol) hydrophilic block and a hydrophobic block composed of either poly(e-caprolactone) (PCL), or poly(L-lactide) (PLA), or poly(lactide-co-glycolide) (PLGA). FIG. 1B shows transmission electron microscopy images of negatively stained nanocarriers. Scale bar=50 nm. FIGS. 1C-D show characterization of nanocarrier size by dynamic light scattering (units=nm). The number-average distribution of nanocarrier diameter (FIG. 1C) and the mean±standard deviation (FIG. 1D) is displayed. Nanocarrier size between formulations was not significantly different. FIG. 1E shows nanocarrier zeta potential determined by electrophoretic light scattering (ELS). For simplicity, mPEG-b-PCL, mPEG-b-PLA, and mPEG-b-PLGA nanocarriers are abbreviated by the hydrophobic blocks, PCL, PLA, or PLGA in panels (C-D), and throughout the rest of the study. Significance was determined by ANOVA with post hoc Tukey multiple comparisons test and a 5% significance level. *p=0.05.

FIGS. 2A-2C show nanocarrier uptake kinetics in vitro. FIG. 2A is an experiment illustration. HEK293T cells were treated with DiI dye-loaded mPEG-b-PCL (“PCL”), mPEG-b-PLA (“PLA”), or mPEG-b-PLGA (“PLGA”) nanocarriers for 2 hours, 24 hours, or 48 hours. FIG. 2B shows the percentage of nanocarrier positive cells (NC+) determined by flow cytometry. The mean % NC+ cells±s.d. (n=3) is displayed. One phase association models (dashed lines) were fit to the data (r2>0.99 in all cases). FIG. 2C shows the rate of nanocarrier uptake. Significance was determined by ANOVA with Tukey's post hoc multiple comparisons test and a 5% significance level. ****p<0.0001.

FIGS. 3A-3F show intracellular accumulation and depletion of U-¹³C-Palmitate tracer. FIG. 3A is a diagram depicting cellular uptake mechanism for the traditional BSA method versus the nanocarrier delivery strategies. FIG. 3B-C show cytotoxicity assessment of cells treated with 50 μM palmitate delivered via BSA or nanocarrier forms (n=4). PBS and 2.5% DMSO (v/v) treatment groups were included as controls. FIG. 3B shows cell proliferation with time. FIG. 3C shows SyTox green staining at 24 hours and 48 hours as an orthogonal toxicity readout. Significance was determined by two-way ANOVA with post hoc Tukey's multiple comparisons test. FIG. 3D shows intracellular pool size of the mass+16 (M+16) species of the U-¹³C-palmitate tracer with time. Inset: M+16 palmitate pool size after BSA-mediated delivery (zoomed in). In all cases, the mean s.d. (n=3) is presented. The dashed lines represent non-linear models of the form y=(y0−y¥)*e−kx fit to the data for each treatment group, where y=the M+16 palmitate (tracer) pool size, x=time (h), y0=the tracer level at t0, y¥=the y value at infinite times (i.e., the plateau), and k is the rate constant (h−1). FIG. 3E shows a statistical analysis of the M+16 palmitate levels at 2 hours. FIG. 3F shows the ratio of intracellular M+16 palmitate at 2 hours (earliest timepoint) with respect to the levels achieved by the BSA method. For FIGS. 3E-F, mean±s.d. (n=3) is presented. Significance was determined by ANOVA with post hoc Tukey's multiple comparisons test. A 5% significance level was used for all statistical tests. *p<0.05; **p<0.01; ****p<0.0001.

FIGS. 4A-4I show that polymeric nanocarriers achieve high intracellular U-¹³C-palmitate accumulation and improved fatty acid labeling consistency. FIG. 4A is an overview of palmitate utilization pathways. FIG. 4B shows the average (n=3) total pool size for the subset of 107 metabolites that differed significantly across groups. The row-wise zscore is displayed. Significance was determined by ANOVA. Hierarchical clustering was performed using the Euclidean distance metric. FIG. 4C shows total label enrichment (%) determined in palmitate (FA 16:0; solid line) and stearic acid (FA 18:0; dashed line) with time. The mean±s.d. (n=3) is presented. FIGS. 4D-G show label enrichment in citric acid cycle intermediates (TCA) and fatty acids (FAs) downstream of palmitate (FA 16:0) at (FIG. 4D) 2 h, (FIG. 4E) 6 h, (FIG. 4F) 24 h, or (FIG. 4G) 48 h following the administration of U-¹³C-palmitate as a BSA complex of as a nanocarrier formulation. This analysis is limited to the subset of TCA intermediates and FAs that are detected using the ion-paired LC-MS method used for metabolomics experiments. In all cases, the mean±s.d. (n=3) is displayed. FIGS. 4H-4I show coefficient of variation determined for the pool size of the M+16 species of each of the five fatty acids measured (FA 16:0, FA 16:1, FA 18:0, FA 18:1, FA 20:1) at (FIG. 4H) 2 hours and (FIG. 4I) 6 hours. The mean±s.d. (n=3) is displayed. A common 30% CV threshold is displayed using a dashed line. Statistical significance was determined via ANOVA with post hoc Tukey's multiple comparisons test. All statistical tests used a 5% significance level. *p<0.05; **p<0.01; ****p<0.0001.

FIGS. 5A-5H show nanocarrier delivery of U-¹³C-palmitate produces complex lipid labeling that correlates with BSA-based methods but exhibits faster kinetics. FIG. 5A is a heatmap summarizing average (n=3) of the total labeled pool size for each lipid. The z-score is presented. Hierarchical clustering was performed using the Euclidean distance metric. FIG. 5B shows principal component analysis (PCA) of the label enrichment at 6 hours. FIG. 5C shows PCA factor loadings. Annotations are provided for the top 10 loadings. FIG. 5D shows label enrichment for the top 10 loadings annotated in (FIG. 5C). FIGS. 5E-H show spearman correlation analysis of complex lipid labeling achieved after delivering U-¹³C-palmitate with BSA versus the specified nanocarrier type. Correlations are presented for (FIG. 5E) 2 h, (FIG. 5F) 6 h, (FIG. 5G) 24 h, and (FIG. 5H) 48 h timepoints. The spearman correlation coefficient (rs) and the approximate, two-tailed p-value are inset within each plot. ****p<0.0001.

FIGS. 6A-6C show nanomaterials are non-toxic to HEK293T cells in vitro. FIG. 6A shows that cell proliferation was monitored by live cell imaging over a 96-hour time course after dosing cells with PBS, 2.5% DMSO, nanocarriers (0.1 mg/mL polymer concentration), or BSA (0.1 mg/mL protein concentration). Cell death was further quantified at (FIG. 6B) 24 h and (FIG. 6C) 48 h via SyTox green staining. Significance was determined via ANOVA with Dunnett's multiple comparisons test and a 5% significance level. ****p<0.0001.

FIGS. 7A-7B show flow cytometry gating strategy for determining nanocarrier uptake by HEK293T cells in vitro. Side scatter area (SSC-A) versus forward scatter area (FSC-A) gating was used to isolate the population of cells from debris. Forward scatter height (FSC-H) versus forward scatter area (FSC-A) gates were set on this cell population to isolate single cells from doublets. Zombie aqua viability stain was used to isolate the population of live cells from that of dead cells. This threshold was set using (FIG. 7A) dead cell controls consisting of a mixture of live cells with heat-treated dead cells (cells in the dead cell control were incubated at 85° C. for 5 minutes then allowed to cool prior to mixing with live cells). The manufacturer recommendation of Zombie Aqua concentration is very high and leads to high viability staining signal, yet a distinct peak is visible for the dead cell population in the event of significant toxicity. A stringent live/dead threshold was set using this toxicity control. The DiI dye gate was set using the (FIG. 7B) PBS-treated cells (background control) to quantify the uptake of nanocarriers by cells with a false positive rate of <1%.

FIG. 8 shows flow cytometry assessment of the cellular uptake of dye-loaded nanocarriers after repeated washing steps. HEK293T cells were harvested for flow cytometry after washing cells once (1×), twice (2×), or three (3×) times with PBS. The plot displays the median fluorescence intensity (MFI) of cells after treatment with DiI-loaded mPEG-b-PLGA nanocarriers for 24 hours. The mean MFI±standard deviation is plotted (n=3). No significant decreases in nanocarrier uptake were found after increasing the washing steps, as determined by ANOVA with post hoc Tukey's multiple comparisons test and a 5% significance level.

FIG. 9 shows depletion of the fully labeled U-¹³C-palmitate from the cell culture medium with time. The total pool size of the mass+16 species (M+16) of palmitate (FA 16:0) measured in the media. The mean±s.d. (n=3) is displayed at each timepoint. The dashed lines represent non-linear models of the form y=(y0−y∞)*e−kx fit to the data for each treatment group, where y=the M+16 palmitate (tracer) pool size, x=time (h), y0=the tracer level at t0, y∞=the y value at infinite times (i.e., the plateau), and k is the rate constant (h−1).

FIG. 10 is a Metabolite Pool Size Heatmap. The average (n=3) total pool size for each measured metabolite. The row-wise zscore is displayed. Hierarchical clustering was performed using the Euclidean distance metric.

FIG. 11 is a histogram of metabolite coefficient of variation (CV) in pooled quality control technical controls. The plot captures the CV distribution of 155 metabolites determined in the pooled QC technical controls (n=7). The dashed line indicates the exclusion threshold of CV>30.0%. Compounds that were excluded from the metabolomics dataset on the basis of this CV filter are summarized in Table 4.

FIG. 12 shows a metabolomics quality control variation assessment. A principal component analysis (PCA) of metabolite pool size measured in the ion-paired (IP) method is presented. Experimental samples are displayed as open circles whereas pooled QC controls are displayed as shaded circles. The percentage of variation accounted for by the first (PC1) and second (PC2) principal components is provided in parentheses. Covariance confidence ellipses are scaled by three standard deviations.

FIG. 13 is a histogram of lipid coefficient of variation (CV) in pooled quality control technical controls. The plot captures the CV distribution of 113 metabolites determined in the QC-NL technical controls (n=8). The dashed line indicates the exclusion threshold of CV≥30.0%. Compounds that were excluded from the metabolomics dataset on the basis of this CV filter are summarized in Table 7.

FIG. 14 shows a lipidomics quality control variation assessment. A principal component analysis (PCA) of metabolite pool size is presented for the experimental samples (open circles) and pooled QC controls (shaded circles) run on the C30 positive lipidomics method used in this study. The percentage of variation accounted for by the first principal component (PC1) and second principal component (PC2) is displayed in parentheses. Covariance confidence ellipses are scaled by three standard deviations.

### DETAILED DESCRIPTION OF THE INVENTION

The use of the term “a” or “an” as used herein in connection with the term “comprising” in the claims and/or the specification may mean “one,” but it also is consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.”

The term “about”, as applied to one or more values of interest, refers to a value that is similar to a stated reference value. “About” is used to indicate that a value includes the inherent variation of error in the composite nanoparticle or composition, of the method being employed to determine the value, or of the variation that exists among different cells, tissues, organs, or subjects. In some embodiments, the term “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction from the stated reference value (unless otherwise stated or made clear from the context). In some embodiments, “about” may mean+/−1-10% of the stated reference value.

Where ranges are given, endpoints are included. Values that are expressed as ranges can assume any specific value or sub-range within the stated ranges in different embodiments of the disclosure.

The term “agent” as used herein refers to any chemical, biological, or pharmacological substance or compound, including but not limited to nanoparticles, small molecules, macromolecules, nucleic acids, peptides, proteins, lipids, carbohydrates, cells, viruses, vectors, or combinations thereof, that may modulate a chemical, biological, or biochemical process, bind to a target molecule, alter gene expression, or produce a therapeutic, diagnostic, or prophylactic effect. The term also encompasses natural, synthetic, or recombinant forms, as well as agents identified through screening or rational design. An agent can comprise an extract made from biological materials, a mixture of biological molecules or macromolecules, a chemical compound, a mixture of chemical compounds, or a mixture of chemical compounds and biological molecules or macromolecules.

The term “amphiphilic material” as used herein is a material that is formed from subunits or monomers that have different hydrophobic or hydrophilic characteristics. These subunits typically are present in at least two blocks of a given character, such as a hydrophobic block and a hydrophilic block. The term “amphiphilic material” as used herein may be a polymeric, non-polymeric, or a polymer blend material.

The term “biodegradable” as used herein refers to a polymer that can degrade chemically and/or biologically within a physiological environment, such as within a cell, tissue, organ, or the body of a subject. A biodegradable polymer can, within the cell, tissue, organ, or the body of a subject, hydrolyze upon exposure to water, or can degrade upon exposure to heat, low pH, enzymatic activity, or other cellular machinery.

The term “cell” as used herein refers to any prokaryotic or eukaryotic individual cell, cell population, cell line, or cell culture.

The term “compound” as used herein refers to all isomers and isotopes of the structure described. “Isotopes” refers to atoms having the same atomic number but different mass numbers resulting from a different number of neutrons in the nuclei.

The term “copolymer” as used herein is where more than one type of repeat unit is present within a polymer. The repeat units forming the copolymer can be arranged as a “block” copolymer, i.e., one or more regions or portions each comprising a first repeat unit (e.g., a first block) and one or more regions each comprising a second repeat unit (e.g., a second block). Such regions or portions of the copolymer each may be generally hydrophilic or generally hydrophobic. In one example, copolymers of the present invention may include tri-block copolymers having a first repeat unit (a first block), a second repeat unit (a second block), and a third repeat unit (a third block); one exemplary triblock copolymer is poly(ethylene glycol)-block-poly(lactide)-block-poly(ethylene glycol) (PEG-b-PLA-b-PEG).

The terms “deliver” and “delivering” as used herein refer to the transfer of the present composite nanoparticle or composition thereof to a physiological site, cell, tissue, or organ. This includes delivery to the intracellular portion of a cell or to the extracellular space of the cell. The delivery of a composite nanoparticle or a composition thereof to a site, cell, tissue, or organ can include an in vitro approach (i.e., delivery to a site, cell, tissue in an artificial environment, such as, in a test tube, reaction vessel, in cell culture, or Petri dish, rather than within an organism), an in vivo approach (i.e., delivery within an organism, such as, within an animal, plant, or microbe, or a cell, tissue, or organ thereof), or an ex vivo approach (i.e., delivery outside of an organism).

The terms “diagnose”, “diagnosing” and “diagnostically” as used herein refer to determining (by one or more individuals) the cause or nature of a problem, situation, or condition in a subject, or a confirmation of a “diagnosis” that includes alternative or other diagnostics, signs, and/or symptoms. A diagnosis may include a test or an assessment of the degree of severity of a condition or disease (e.g., “mild,” “moderate,” or “severe”), current state of progression of a condition or disease (e.g., “early”, “middle,” or “late” stage), or a comparative assessment to an earlier diagnosis. A diagnosis may include a “prognosis” that is a future prediction of the progression of a condition or disease.

The term “effective amount” as used herein means an amount sufficient to have the desired effect or biochemical result on or in the subject, cell, tissue, or organ. The effective amount may depend on various factors related to the subject, cell, tissue, or organ, including age, physical condition, size, and weight; the frequency of delivery or administration or treatment of the composite nanoparticle or composition; a reasonable benefit/risk ratio, and/or the mode of delivery or administration. These factors are well known to those of ordinary skill in the art and can be determined with routine experimentation. The term “diagnostically effective amount” as used herein refers to an amount sufficient to determine (by one or more individuals) the cause or nature of a problem, situation, or condition in a subject, or a confirmation of a diagnosis that includes alternative or other diagnostics, signs, and/or symptoms

The term excipient as used herein refers to any pharmaceutically acceptable material or substance that is included in a composition with one or more active molecules that is not itself active but allows the one or more active molecules or organisms to retain its activity. The choice of excipient will largely depend on factors such as the mode of administration, the effect of the excipient on solubility and stability, and the nature of the dosage form. Excipients may serve various roles including, but not limited to, acting as solvents, carriers, diluents, dispersion media, fillers, binders, stabilizers, coatings, preservatives, antibacterial and antifungal agents, solubilizers, buffers, emulsifiers, surfactants, disintegrants, isotonic and absorption delaying agents, or agents that modify the release profile, bioavailability, or stability of the active ingredient.

Excipients are often used to formulate biologically active molecules or organisms to maintain structural integrity and/or biological activity during storage and administration and may include sugars, salts, polymers, amino acids, lipids, and other suitable compounds. Examples of an excipient include one or more of water, saline, phosphate-buffered saline, dextrose, glycerol, ethanol, and the like, as well as combinations thereof, and may include isotonic agents, for example, sugars, sodium chloride, or polyalcohols such as mannitol or sorbitol in the composition.

The terms “hydrophilic” as used herein describes a physical property of a molecule that is capable of hydrogen bonding with a water (H₂O) molecule and is soluble in water and other polar solvents. Conversely, the term “hydrophobic” as used herein describes a physical property of a molecule that is repelled from a mass of water and can be referred to as “nonpolar,” or “apolar”. Exemplary hydrophobic compounds include, but are not limited to fatty acids, fatty acid derivatives, and lipid analogs.

The term “nanocarrier” is used herein to refer to a particle carrier (of an active agent or molecule) with dimensions at the nanoscale (10⁻⁹ m) that can load, encapsulate, and/or integrate the active agent or molecule within its core or internal layer.

The terms “nanoparticle” and “composite nanoparticle” are used interchangeably herein to refer to a nanocarrier that is loaded with a hydrophobic compound.

The term “pharmaceutically acceptable” as used herein means compounds and compositions that are suitable for use in contact with the tissues of human beings and/or animals without excessive adverse event or problem and commensurate with a reasonable benefit/risk ratio.

The term “pharmaceutically acceptable carrier” as used herein refers to any carrier, diluent or excipient, or compound (other than the composite nanocarriers disclosed herein) that is compatible with other ingredients of the formulation and compatible with the intended route of administration. A pharmaceutically acceptable carrier is a non-toxic, inert substance or mixture that is suitable for the formulation, delivery, or administration of an active agent to a subject, and that does not interfere with the activity or stability of the agent. The carrier may facilitate solubilization, stabilization, preservation, or delivery of the active ingredient and is compatible with physiological conditions. Examples of pharmaceutically acceptable carriers include, but are not limited to, water, saline, buffers, sugars, polyols, lipids, polymers, surfactants, oils, emulsifiers, and biodegradable microspheres or nanoparticles. The carrier is selected based on the intended route of administration and the nature of the active agent (e.g., a hydrophobic compound). Examples of common pharmaceutical carriers include phosphate buffered saline solution, water, emulsions (e.g., oil/water or water/oil emulsions), and various types of wetting agents. The compositions also can include stabilizers and preservatives. For more examples of carriers, stabilizers, and preservatives, see e.g., Martin, Remington's Pharmaceutical Sciences, 15th Ed. Mack Publ. Co., Easton, Pa. (1975).

The term “pharmaceutical composition” refers to a preparation comprising at least one biologically active agent (e.g., a hydrophobic compound), optionally in combination with one or more pharmaceutically acceptable carriers, excipients, or diluents, formulated for administration to a subject for therapeutic, prophylactic, or diagnostic purposes in vivo or ex vivo. The composition can be suitable for administration via any appropriate route, including but not limited to oral, intravenous, subcutaneous, intramuscular, intranasal, topical, or intrathecal routes. The components of the pharmaceutical composition are selected to preserve the stability, activity, and bioavailability of the biologically active agent under conditions of storage and use.

The term “polydispersity index” as used herein is a ratio that describes the homogeneity of the particle size distribution of a system. A small value indicates a narrow particle size distribution.

The term “polymer” as used herein refers a molecular structure comprising one or more repeat units (monomers), connected by covalent bonds. The repeat units can all be identical, or in some cases, there can be more than one type of repeat unit present within the polymer. In some cases, additional moieties can be present in the polymer, for example tracer moieties such as those described herein. A hydrophilic polymer generally attracts water, and a hydrophobic polymer generally repels water.

The term “polymer blend” as used herein refers to combinations of amphiphilic polymers, hydrophilic polymers, hydrophobic polymers, biodegradable polymers, or functionalized polymers, and may optionally include additional non-polymeric components such as lipids, surfactants, or small molecules. The relative ratios of polymers in a polymer blend may be selected to tune nanoparticle properties including size, stability, payload loading, release kinetics, and biological interactions.

The term “subject” as used herein refers to any organism to which a composite nanoparticle or composition as disclosed herein may be administered, e.g., for experimental, diagnostic, prophylactic, and/or therapeutic purposes. Typical subjects include animals (e.g., mammals such as mice, rats, rabbits, non-human primates, and humans) and/or plants. In some embodiments, humans are suitable subjects. Human subjects may be of any gender and at any age or stage of development. In some embodiments, the subject is receiving or intended to receive a treatment, diagnosis, or prophylaxis, including administration of a compound or pharmaceutical composition. The terms encompass both healthy individuals and those suffering from a disease, disorder, condition, or injury, regardless of the stage or severity.

The term “tracer” as used herein refers to a labeled molecule that behaves like or mimics a natural molecule in its location, pathway, or action in a biological or environmental system. Exemplary types of labeling include isotopic and fluorescent labeling of a molecule.

The term “uniformly labeled” as used herein refers to a compound in which every atom in the compound has been replaced by an isotope of that atom.

Disclosed herein are composite nanoparticles that include a nanocarrier and a hydrophobic compound loaded in the nanocarrier. The nanocarrier may be biodegradable.

In some embodiments, the nanocarrier is an amphiphilic material. In some embodiments, the amphiphilic material is a poly(ethylene glycol)-block-poly(ε-caprolactone) copolymer (PEG-b-PCL), a poly(ethylene glycol)-block-poly(L-lactide) copolymer (PEG-b-PLA), or a poly(ethylene glycol)-block-poly(lactide-co-glycolide) copolymer (PEG-b-PLGA). The poly(ethylene glycol) block of any of the biodegradable amphiphilic materials may be terminated with a hydroxyl group (—OH), a methyl group (—CH₃), a phosphate group (—PO₄), an amine group (—NH₂), a carboxyl group (—CO₂H), a thiol group (—SH), an aldehyde group (—CHO), a vinyl group (—C₂H₃), an epoxide group, a malemide group, or an azide group (N₃). In one embodiment, the poly(ethylene glycol) block of the biodegradable amphiphilic materials may be methoxy poly(ethylene glycol), i.e., “mPEG.

Representative materials used in the Examples set forth below are summarized in Table 1. The molecular weight of the hydrophilic block (e.g., 2000 g/mol) and hydrophobic block (10,000 g/mol), as well as the hydrophilic weight fraction of the copolymer, can be varied to adjust nanocarrier physical properties. In some embodiments, between about 5% and about 25% of the molecular weight of the nanocarrier is the hydrophilic block portion of the nanocarrier; or between about 10% and about 20% of the molecular weight of the nanocarrier is the hydrophilic block portion. In one embodiment, about 17% (e.g., 16.7%) % of the molecular weight of the nanocarrier is the hydrophilic block portion of the nanocarrier.

TABLE 1
Polymer summary.

| Polymer | Mw^{†} | Mn^{†} | PDI^{†} |
|---|---|---|---|
| mPEG₂ₖ-b-PCL₁₀ₖ | 12,200 | 11,200 | 1.09 |
| mPEG₂ₖ-b-PLA₁₀ₖ | 12,800 | 12,800 | 1.09 |
| mPEG₂ₖ-b-PLGA₁₀ₖ | 12,100 | 12,100 | 1.09 |

^{†}Polydispersity index (PDI) determined by gel permeation chromatography (GPC).

The “m” in the “mPEG” block means that the PEG block is a methoxy-terminated poly(ethylene glycol).

Various forms of mPEG-b-PLA, mPEG-b-PLGA, mPEG-b-PCL are known. These copolymer nanocarriers can be synthesized using established methods; and there are commercial options for obtaining the polymers. The properties of the copolymer nanocarriers (e.g., size) can influence the physical properties of the nanostructures. Also, the hydrophobic block (PCL, PLA, PLGA) may influence the rate of tracer release inside a cell. As described herein, the properties of the polymers can be adjusted to impact the physical properties of the nanostructures.

The nanocarriers described herein are characterized by a micellar nanoarchitectures (micelles). Micelles are colloidal aggregates of amphipathic molecules that are formed at a well-defined concentration known as the critical micelle concentration. Micelles are oriented with the hydrophobic portions of the lipid molecules at the interior of the micelle and the hydrophilic portions at the exterior surface, exposed to water.

The nanocarriers of the present invention are capable of loading (e.g., encapsulating or including as part of the nanocarrier) a hydrophobic compound that enables tracking the movement, incorporation, and metabolism of the hydrophobic compound within a subject, tissue, and/or cell. Specifically, the nanocarrier (micelle) is capable of transporting a hydrophobic tracer through a biofluid and delivering a hydrophobic tracer to cells where they facilitate the effective intracellular release of the hydrophobic tracer (e.g., through degradation of the nanocarrier) and the participation of the hydrophobic tracer in metabolic reactions and processes.

In some embodiments, the hydrophobic compound that is loaded in the nanocarrier is an isotopically labeled hydrophobic compound. The isotopic label of the hydrophobic compound may be a stable isotope label, such as carbon-13 (¹³C). Further, the stable isotope label of the hydrophobic compound includes any of hydrogen-2 (²H), nitrogen-15 (¹⁵N), or oxygen-18 (¹⁸O).

The hydrophobic compound of the composite nanoparticle may be palmitate (C₁₆H₃₁O₂), a salt or ester of palmitic acid, oleate (C₁₈H₃₃O₂), or a salt or ester of oleic acid. The palmitate may be ¹³C-palmitate, or more specifically, U-¹³C-palmitate. U-¹³C-palmitate is palmitate that is uniformly labeled with ¹³C. In some embodiments, the U-¹³C-palmitate is provided in an encapsulated concentration range from about 2 mM to about 10 mM. In specific embodiments, the encapsulated concentration of U-¹³C-palmitate is 0.05 mM, 2 mM, or 5 mM.

In one embodiment, the nanocarrier additionally includes a co-loaded hydrophobic fluorescent dye for multiple purposes (e.g., a fluorescent dye that is loaded on the nanocarrier along with the stable isotope labeled hydrophobic compound), including for facilitating the selective purification of nanocarrier formulations and for assessing nanocarrier biodistribution in organs and/or internalization by cells. The fluorescent dye may be DiI, also known as “DiIC18(3)”. DiI is a well-known long-chain carbocyanine dye.

Further, the composite nanoparticles have an average diameter in a range between about 20 nm and about 400 nm; or the average diameter of the composite nanoparticle may range from about 20 nm to about 50 nm; or the average diameter of the composite nanoparticle may range from about 30 nm to about 45 nm. The average diameter of a plurality of composite nanoparticles may be about 400 nm or less, 200 nm or less, 100 nm or less, 50 nm or less, 40 nm or less, 30 nm or less, 20 nm or less, or 10 nm or less. In some embodiments, the composite nanoparticles have a polydispersity index (PDI) that ranges between 1.50 and 0.1; or the PDI may range between about 1.3 and 1.0.

The composite nanoparticles may have a surface zeta potential in the range of about −50 mV to about +50 mV; or a surface zeta potential may be in the range of about −20 mV to about −10 mV.

To make the composite nanoparticle of the present invention, the hydrophobic compound can be loaded or combined with the copolymer nanocarriers in two different ways to form the inventive composite nanoparticle. One is during the self-assembly process itself, i.e., where the hydrophobic compounds are loaded into the nanocarriers during the self-assembly of the nanocarrier, and the other is through integrating or embedding the hydrophobic compounds into the already-formed nanocarriers. After the hydrophobic compound is combined with one of the copolymers, the hydrophobic compound is located on the interior of the micelle. The resulting composite nanoparticle can be further subjected to one or more post-formation processing, modification, or formulation steps, including but not limited to purification techniques (e.g., chromatography, dialysis, tangential flow filtration, or centrifugation), size-altering or homogenization techniques (e.g., extrusion, sonication, or filtration), buffer exchange, adjustment of pH or ionic strength, addition or removal of salts, surfactants, stabilizers, cryoprotectants, targeting ligands, or other excipients, concentration or dilution, sterilization, and physicochemical characterization.

In embodiments where the composite nanoparticle comprises a liposomal or lipid nanoparticle (LNP) structure, the methods of manufacture are conceptually analogous to those described for polymeric nanocarriers, with differences primarily arising from the choice of amphiphilic materials and the mechanisms governing self-assembly. Liposomal and LNP formulations may be prepared using known techniques such as thin-film hydration, ethanol or solvent injection, microfluidic mixing, rapid aqueous dilution, or combinations thereof, wherein phospholipids, ionizable lipids, cationic lipids, neutral lipids, cholesterol, or mixtures thereof self-assemble into vesicular or lipid nanoparticle structures upon contact with an aqueous phase. In such embodiments, the hydrophobic compound may be incorporated during particle formation by co-dissolution with lipid components prior to self-assembly, or may be introduced into pre-formed liposomal or LNP structures via post-assembly loading or partitioning. Following formation, liposomal and LNP-based composite nanoparticles may be subjected to the same or similar post-formation processing, formulation, and optimization steps as described for polymeric nanocarriers, including purification, size modification, buffer exchange, adjustment of pH or ionic strength, excipient addition or removal, and characterization. Accordingly, the inventive composite nanoparticle and tracer delivery approach is not limited by the specific self-assembling material system employed, and encompasses polymeric, lipid-based, and hybrid polymer-lipid nanoparticles prepared using analogous manufacturing principles.

Dosing is based on the palmitate concentration in the composite nanoparticle. For example, with in vitro studies, a 50 μM concentration of U-¹³C-palmitate (i.e., a 50 μM working/in-well concentration in cell-based assays) may be used. Such a 50 μM dose is considered to be a low dose that is safe and non-toxic (because palmitate can become toxic at higher concentrations) and provides a good signal in metabolomics and lipidomics studies (i.e., good labeling). Dosing at a concentration in a range from about 50 uM up to about 200 uM may be used. Further, dosing should be at a concentration that is under the cell-type specific toxicity threshold with in vivo studies, a 5 mM U-¹³C-palmitate concentration, administered intravenously (i.v.), may be used. In one embodiment, this composition is i.v.—administered in a 200 μL bolus followed by a constant infusion for 4-6 hours. For in vitro applications, concentrations above 200 μM becomes toxic (but this toxicity threshold depends on cell type). Dosing for in vitro cell proliferation studies (monitored by live cell imaging) may be at a 0.1 mg/mL polymer concentration. Thus, the composite nanoparticle provides good labeling with palmitate at a low concentration.

Nanocarriers may be prepared from mPEG₂ₖ-b-PCL₁₀ₖ, mPEG₂ₖ-b-PLA₁₀ₖ, and mPEG₂ₖ-b-PLGA₁₀ₖ polymers (Table 1) by the method of self-assembly, e.g., using a confined impingement jets mixer. More specifically, the copolymer nanocarriers and hydrophobic compound may be dissolved in an organic solvent (e.g., tetrahydrofuran), mixed by the impingement jets, e.g., impinged against a aqueous stream of phosphate buffered saline (PBS) or water into an aqueous reservoir (e.g., PBS or water), and incubated (e.g., in a desiccator overnight) to remove any residual organic solvent. In another example, potassium U-¹³C-palmitate can be dissolved in DMSO and integrated into self-assembled polymeric nanocarriers using established methods. In another example, the polymeric nanocarriers can be purified further using size exclusion chromatography, LH-20 chromatography, and/or dialysis.

Aside from nanoprecipitation and flash nanoprecipitation techniques, the described nanocarriers can also be prepared using alternative methods. These methods include cosolvent precipitation, thin film hydration, emulsion, and/or other related procedures that are commonly used to form nanoparticles from polymer amphiphiles.

The size of the nanocarriers produced can also be further modified using extrusion techniques employing filters of defined size thresholds, as needed.

The resulting nanocarrier formulations can be purified by various chromatography techniques, centrifugation-based procedures, and/or dialysis techniques, as needed.

Some embodiments of the invention are compositions that include the disclosed composite nanoparticles These compositions may include one or more of the composite nanoparticles described herein. In one embodiment, the composition may include: (1) one of the copolymers poly(ethylene glycol)-block-poly(ε-caprolactone) copolymer (PEG-b-PCL), poly(ethylene glycol)-block-poly(L-lactide) copolymer (PEG-b-PLA), or poly(ethylene glycol)-block-poly(lactide-co-glycolide) copolymer (PEG-b-PLGA), and (2) a hydrophobic compound that incorporates a stable isotope label (e.g., carbon-13, hydrogen-2, nitrogen-15, oxygen-18, etc.).

In some embodiments, the composite nanoparticles of the composition each can be substantially the same shape (e.g., spherical), size, and/or surface charge (zeta potential). The size, surface charge, and/or shape of the composite nanoparticles of the composition can be selected based on their application or method of use. In other embodiments, the diameter of no more than 25% of the composite nanoparticles varies from the mean particle diameter by more than 150%, 100%, 75%, 50%, 25%, 20%, 10%, or 5% of the mean particle diameter. In yet other embodiments, the surface zeta potential of no more than 25% of the composite nanoparticles varies from the mean particle surface potential by more than 150%, 100%, 75%, 50%, 25%, 20%, 10%, or 5% of the mean particle surface potential.

In some cases, no more than about 5% of the composite nanoparticles of the composition have a dimension greater than about 5%, about 3%, about 1%, about 0.3%, about 0.1%, about 0.03%, or about 0.01% greater than the average dimension of the composite nanoparticles.

Compositions comprising composite nanoparticles of the present invention can be combined with pharmaceutically acceptable carriers to form a pharmaceutical composition. The character of the nanocarriers can be chosen based on the route of administration, the location of the target tissue, the hydrophobic compound being delivered, and the time course of delivery. More specifically, the present pharmaceutical compositions may include (a) a plurality of composite nanoparticles in which each composite nanoparticle of the plurality of composite nanoparticles includes an amphiphilic material and a hydrophobic compound, and the hydrophobic compound includes a stable isotope label; and (b) a pharmaceutically acceptable carrier. In particular, the compositions may include any of the compositions described herein, and a pharmaceutically acceptable carrier.

The pharmaceutical compositions that are used in the methods described herein may be formulated in any form that is appropriate for administration to subjects, animals, tissues, organs, or cells.

The above-described composite nanoparticles and compositions may be used in methods for assessing lipid metabolism or as a component of clinical or diagnostic methods or assays for assessing abnormalities or differences in lipid metabolism that may underlie a disease or health condition. More specifically, the composite nanoparticles and compositions are useful in methods of delivering a hydrophobic compound loaded in a nanocarrier to tissues and cells. The hydrophobic compound may be used as a tracer and/or is metabolism-altering to a cell.

Provided herein are methods of using the disclosed composite nanoparticles and compositions. In one aspect, various methods include following a hydrophobic compound through a biochemical pathway in a cell, including providing the cell, providing the composite nanoparticle or composition, delivering an effective amount of the composite nanoparticle to the cell, and following the hydrophobic compound through the biochemical pathway in the cell. For example, the biochemical pathway may be central carbon metabolism, lipid biosynthesis, lipid remodeling, lipid transport, and any associated pathway involving the hydrophobic compound or products formed through the participation of the hydrophobic compound in one or more biological reactions.

In particular embodiments, the composite nanoparticles are used to deliver an isotope-labeled hydrophobic compound to cells allowing for the metabolism of the delivered payload to be traced within biological environments, which is useful for understanding metabolic phenomena and may also provide use for clinical diagnostics that assess biotransformations of a hydrophobic tracer (e.g., lipids, etc.). The composite nanoparticles also are useful in methods for detecting differences or changes in the metabolism of the loaded hydrophobic compound, as well as other preclinical and/or clinical applications where monitoring the transport of a labeled hydrophobic compound is desired.

Another aspect of the invention is a method of tracking internalization of a hydrophobic compound in a cell, including the steps of providing the cell, providing the inventive composite nanoparticle or composition, delivering an effective amount of the composite nanoparticle to the cell, and tracking internalization of the hydrophobic compound by the cell.

Yet another aspect of the invention is a method of tracking distribution of a hydrophobic compound in a biological organ or tissue, including the steps of providing the biological organ or tissue, providing the inventive composite nanoparticle or composition, delivering an effective amount of the composite nanoparticle to the biological organ or tissue and tracking distribution of the hydrophobic compound in the biological organ or tissue.

For example, the composite nanoparticles and compositions disclosed herein may be used in liquid chromatography-mass spectrometry (LC-MS) based metabolomics and/or lipidomics experiments. These studies use mass spectrometry to follow the incorporation of the 13C carbon into biological metabolites and lipids after the U-¹³C-palmitate tracer is delivered into cells.

Furthermore, the composite nanoparticles and compositions disclosed herein may be used as a clinical diagnostic tool, or as a means of tracking the hydrophobic compounds (for example, in a pharmacokinetics study). For example, the composite nanoparticle and compositions disclosed herein may be used in a method of diagnosing a condition or a disease in a subject, including the steps of providing the inventive composite nanoparticle or composition, administering a diagnostically effective amount of the composite nanoparticle or composition to the subject, or to a cell of tissue of the subject, and tracking the movement, incorporation, or metabolic effect of the composite nanoparticle in the subject, cell, or tissue.

The pharmaceutical composition can be administered to a subject and used in the diagnosis or treatment of the subject. The composition may be formulated for administration intravenously, intra-arterially, orally, transdermally, transmucosally, intraperitoneally, intracranially, intraocularly, epidurally, intrathecally, topically, by enema, by injection, by pulmonary route or by infusion.

Differing administration dosages and routes of administration can be determined for optimal administration of the present composite nanoparticles and compositions.

In one embodiment, if a hydrophobic compound (e.g., palmitate) is to be delivered as part of a medical treatment to a subject, then the composite nanoparticles and compositions disclosed herein may be used in administering that hydrophobic compound to that subject.

The following examples are offered for illustrative purposes only. The examples provided are not intended to limit the scope of the present invention in any way. Various modifications of the invention in addition to those shown and described herein will become apparent to those skilled in the art from the foregoing description and the following examples and fall within the scope of the appended claims.

### EXAMPLES

### Example 1: Physicochemical Characterization of Polymeric Nanocarriers for Delivering Hydrophobic Stable Isotope Labeled Tracers

Self-assembling polymeric micelles encapsulating hydrophobic stable isotope-labeled cargo were developed. The micelle morphology was of particular interest for applications in metabolomics and lipidomics due to its robust self-assembly and small size that permits rapid cellular internalization. Polymeric nanocarriers (NCs) were chosen due to their numerous advantages over more traditional lipid systems. These advantages include greater stability in vivo, minimalistic composition (i.e., no requirements for common additives such as cholesterol for enhanced stability, etc.), improved cargo loading efficiencies, and high customizability (i.e., surface functionalization for targeted drug delivery, etc.). Importantly, polymeric materials avoid confounding effects that would be introduced by carrier materials of liposomes and lipid nanoparticles. Many lipid systems are formulated with materials that would directly modulate fatty acid pools of interest in metabolism studies. For example, liposomal preparations commonly use 1,2-distearoyl-sn-glycero-3-phosphorylethanolamine (DSPE), a saturated phospholipid derived from phosphatidylethanolamine (PE) that contains stearic acid acyl chains; stearic acid is often an analyte of interest in fatty acid tracing experiments. Its inclusion as a carrier material would alter the unlabeled stearic acid pool within the cell, thereby altering biological interpretations across treatment conditions. Similar issues would also arise with dipalmitoylphosphatidylcholine (DPPC), Distearoylphosphatidylcholine (DSPC), dioleoylphosphatidylcholine (DOPC), 1-palmitoyl-2-oleoyl-sn-glycero-3-phosphocholine (POPC), and other common lipid carrier materials.

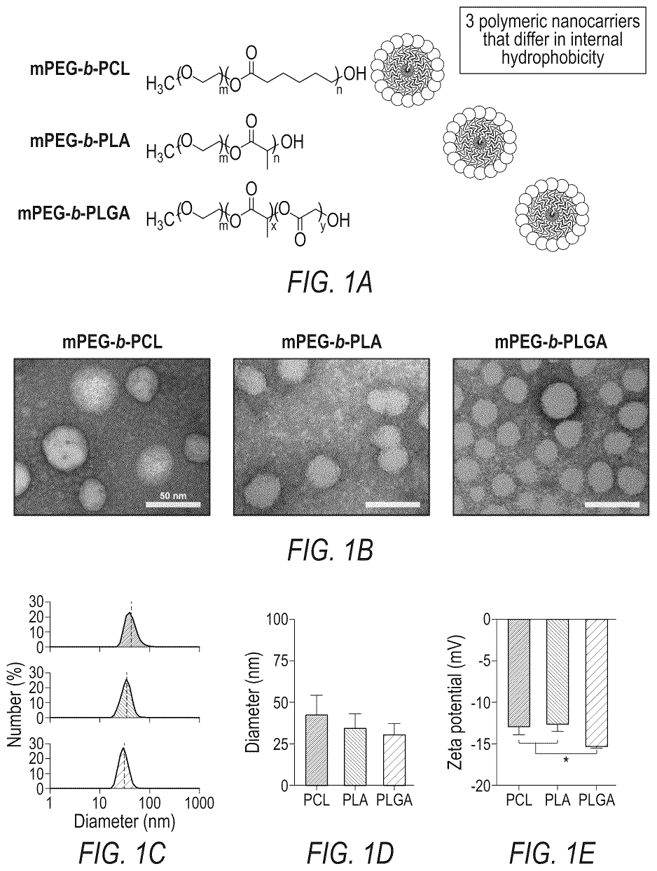

Development proceeded with three different self-assembling linear block copolymer amphiphiles (FIG. 1A). Each polymer type has a fixed hydrophilic weight fraction that promotes the formation of micelle nanocarriers of similar size and charge (Table 2). Methoxy-terminated poly(ethylene glycol) having a molecular weight of 2,000 g/mol was used as the hydrophilic block, which when assembled into a spherical array, presents methyl groups at the nanocarrier surface (FIG. 1A). This methoxy surface chemistry enhances circulation time by minimizing protein adsorption and opsonization and helps reduce the rate of clearance after intravenous (i.v.) administration in vivo. This is an important design consideration for our work since it is often of interest to administer SIL tracers via the intravenous route, wherein reduced biofouling improves the pharmacokinetics of the nanocarrier and its encapsulated cargo.

The block copolymers differed in the chemical composition of their 10,000 g/mol hydrophobic block, which consisted of either poly(ε-caprolactone) (PCL), or poly(L-lactide) (PLA), or poly(lactide-co-glycolide) (PLGA) (FIG. 1A). These are well-established chemistries that are biodegradable but differ in hydrophobicity. The strength of the hydrophobic core influences the rate of intracellular cargo release after the nanocarriers are endocytosed by cells. PCL is the most hydrophobic of the three polymer types used in this study, followed by PLA, whereas PLGA is the least hydrophobic. While similar to PLA, PLGA is less hydrophobic due to it being a copolymer of lactic acid and glycolic acid (here, present at a 1:1 ratio). Glycolic acid contains a greater number of hydroxyl groups per carbon atom that enhances its interaction with water. Testing a small range of similar, but distinct internal chemistries was performed to understand the impact of the intracellular release rate on metabolite labeling kinetics.

TABLE 2
Summary of nanocarrier physical and chemical properties†

| | | | | | Zeta |
|---|---|---|---|---|---|
| | | | Diameter | | potential |
| Nanocarrier | Polymer | Cargo | (nm)^{†} | PDI^{†} | (mV)^{††} |
| PCL | | DiI dye | 42.6 ± 11.7 | 0.08 | −13.0 ± 0.9 |
| PCL +palm | mPEG₂ₖ- | U-¹³C- | 42.1 ± 11.6 | 0.08 | −16.6 ± 0.7 |
| | b-PCL₁₀ₖ | palmitate | | | |
| PLA | | Dil dye | 34.7 ± 8.4 | 0.06 | −12.7 ± 0.8 |
| PLA +palm | mPEG₂ₖ-b- | U-¹³C- | 31.2 ± 10.2 | 0.10 | −15.4 ± 0.7 |
| | PLA₁₀ₖ | palmitate | | | |
| PLGA | mPEG₂ₖ-b- | Dil dye | 30.7 ± 6.5 | 0.05 | −15.3 ± 0.2 |
| PLGA | PLGA₁₀ₖ | U-¹³C- | 30.2 ± 6.1 | 0.04 | −16.9 ± 1.2 |
| +palm | | palmitate | | | |

^{†}Number average diameter and polydispersity index (PDI) determined by dynamic light scattering (DLS).
^{††}Zeta potential determined by electrophoretic light scattering (ELS).

Nanocarriers were prepared by flash nanoprecipitation. The resulting nanostructures had a spherical morphology, as characterized by transmission electron microscopy (FIG. 1B). The size and zeta potential of each colloidal suspension was characterized by dynamic light scattering (DLS) and electrophoretic light scattering (ELS). The nanocarriers were monodisperse (FIG. 1C), with a PDI<0.3 in all cases (Table 2). The mean diameter ranged from 30-45 nm (Table 2), and differences in nanocarrier size between formulations were not significant (FIG. 1D).

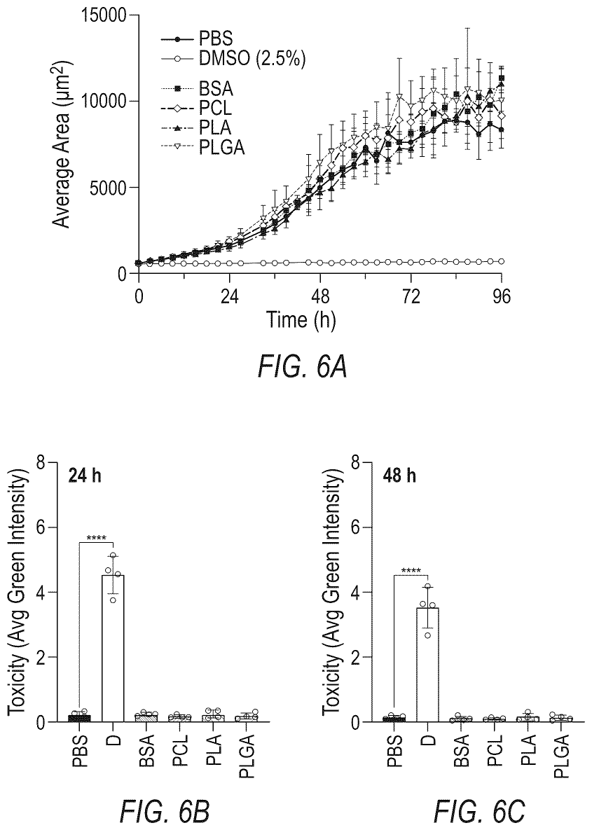

Electrophoretic light scattering demonstrates that all nanocarriers had a weakly anionic surface charge with a mean zeta potential in the −12 mV to −20 mV range (Table 2). The surface charge of the mPEG-b-PLGA micelles (−15.3±0.2 mV) was slightly greater than that of the mPEG-b-PLA (−12.7±0.8 mV) and mPEG-b-PCL micelles (−13.0±0.9 mV) (FIG. 1E). Each formulation was non-toxic to HEK293T cells, as differences in neither proliferation (FIG. 6A) nor cytotoxicity (FIG. 6, B-C) were observed between cells treated with PBS versus nanocarriers over a multiday time course.

### Example 2: Cells Rapidly Internalize Polymeric Nanocarriers

Common methods to deliver SIL lipid tracers in vitro rely on membrane-embedded transporters for cellular entry, such as CD36 (fatty acid translocase). Deficiencies in CD36 produce significant reductions in fatty acid (FA) uptake in various tissues, whereas knocking out this receptor in mice results in a 50% decrease in FA uptake by adipose and muscle tissue. In contrast, tracer delivery using nanocarriers proceeds through endocytosis mechanisms that are generally well characterized for various polymeric and lipid nanoparticle systems. Nanocarriers traffic through endosomes and lysosomes, destabilize within these acidic compartments, and material interactions with compartment membranes promotes intracellular cargo release.

The micelle morphology selected for development has a diameter of less than 50 nm (Table 2). This particle size is capable of permitting rapid internalization.

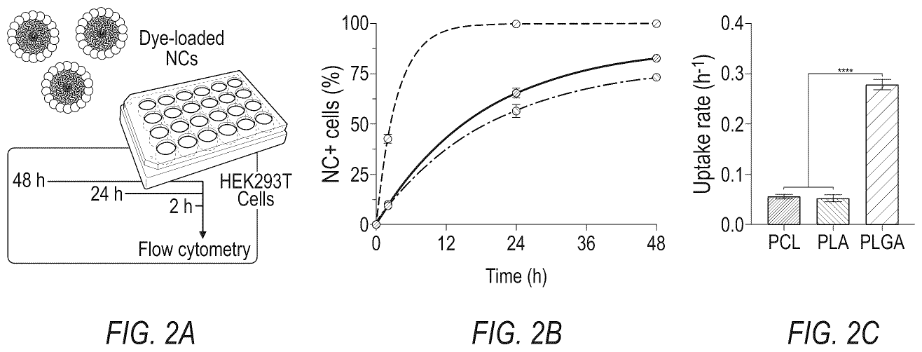

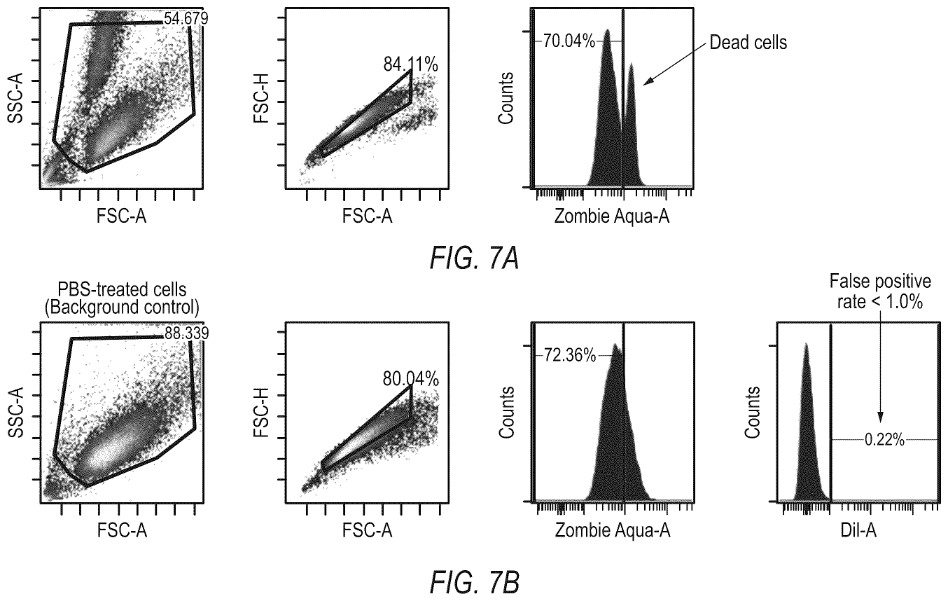

To understand the time scale of cellular entry, dye-loaded nanocarriers were prepared and the extent of their uptake by cells was examined at multiple timepoints over 48 h in vitro. Human embryonic kidney cells expressing a mutant form of the SV40 large T antigen (HEK293T cells) were used as a model cell type. Cells were treated with dye-loaded nanocarriers for 2 h, 24 h, or 48 h prior to harvesting cells for flow cytometry analysis (FIG. 2A). Nanocarrier uptake was quantified in the population of live cells using the fluorescence of the encapsulated DiI cargo (FIG. 7, A-B).

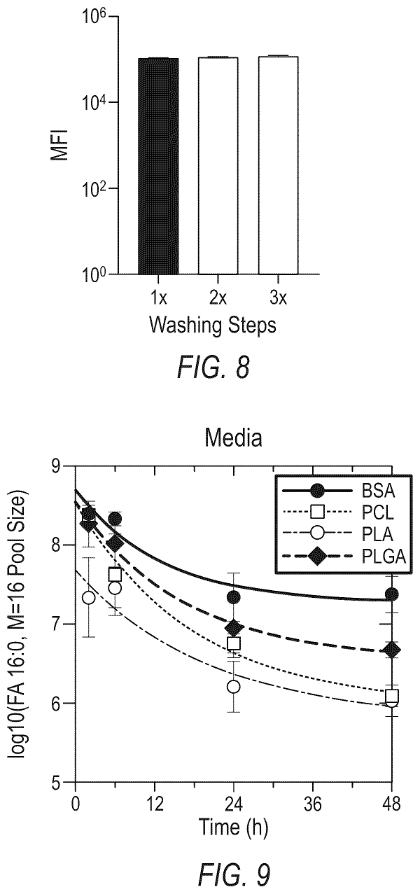

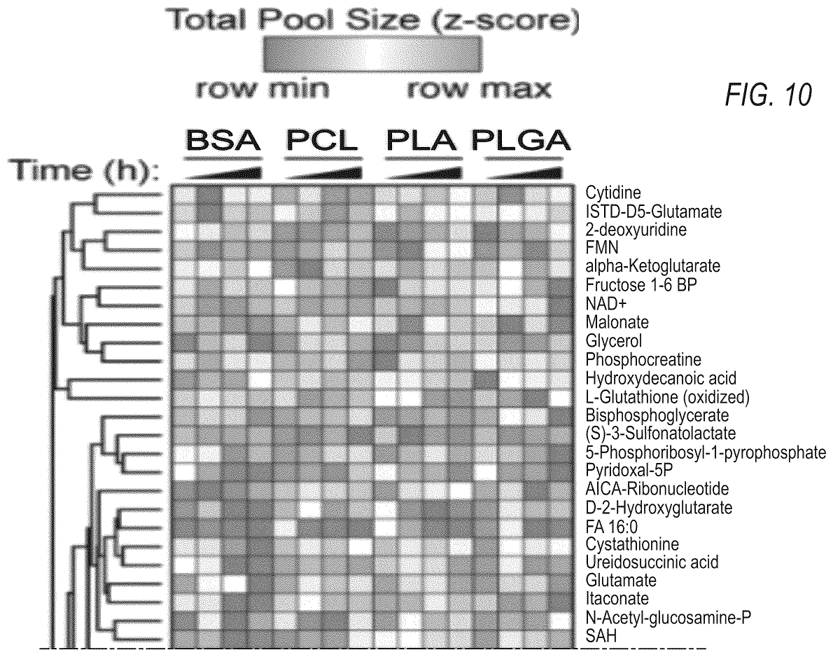

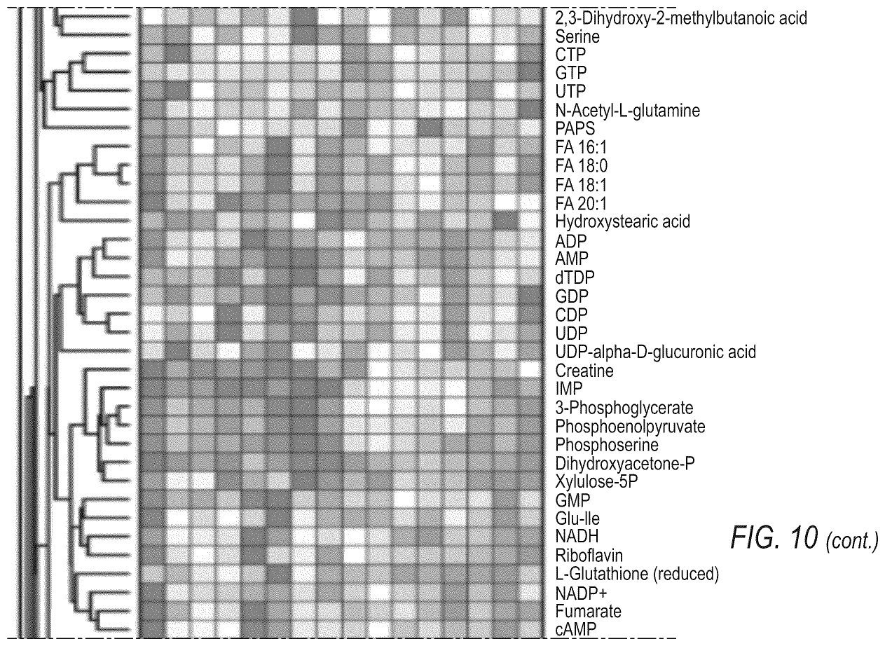

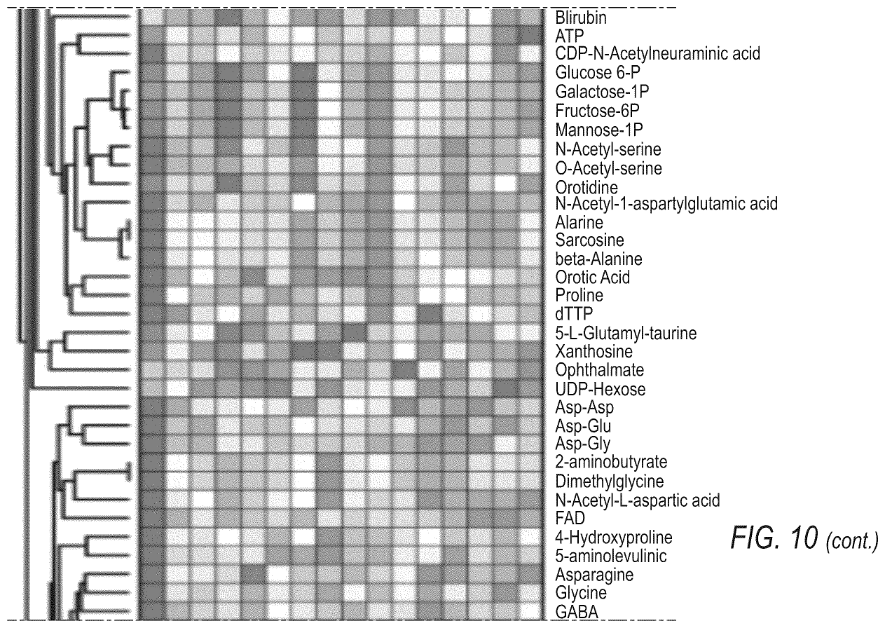

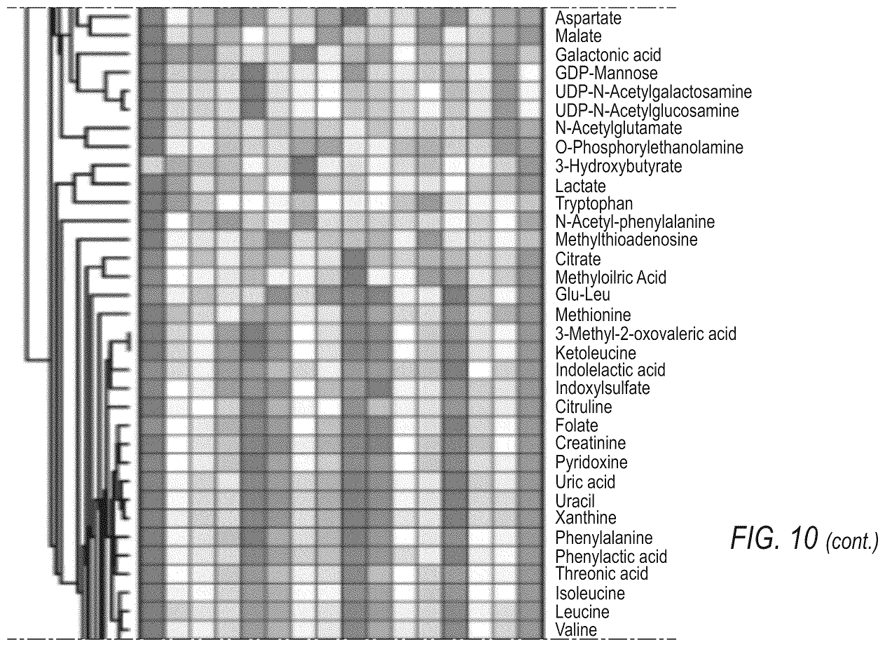

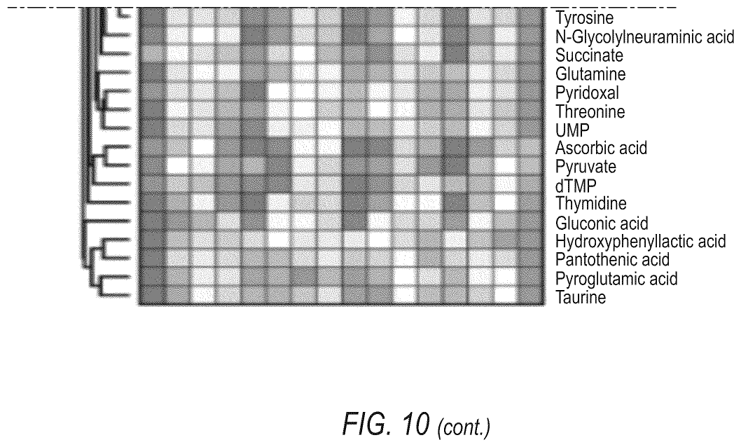

It was ensured that the washing step of the cell harvesting procedure was sufficient to minimize any confounding effects that would inflate nanocarrier uptake levels. The purpose of this assay is to provide a basic understanding of the rate of nanocarrier internalization by HEK293T cells and there should be minimal contributions from nanocarriers that are weakly associated with the cell surface but not yet endocytosed. These preliminary studies demonstrate the median fluorescence intensity (MFI) does not decrease with additional washing steps (FIG. 8). This suggested that washing cells once with PBS is sufficient to remove any contributions from surface-associated nanocarriers, and that this assay thereby provides a reliable measurement of nanocarrier uptake.

Nanocarrier internalization was detected by 2 h (FIG. 2B). The percentage of nanocarrier positive (NC+) cells exceeded 9% in all cases (FIG. 2B). This confirmed HEK293T cells rapidly internalize each nanocarrier type used in this work. Cellular internalization gradually increased with time (FIG. 2B). Greater than 70% of the cells were found to internalize nanocarriers by 48 h (FIG. 2B). While the differences in nanocarrier size were not statistically significant (FIG. 1D), differences in this physical property did appear to influence the cellular internalization rate at 2 hours. The structures having the smallest diameter, nanocarriers self-assembled from mPEG-b-PLGA (“PLGA”) (30.7±6.5; Table 2), were internalized at the greatest rate (FIG. 2C). Nanocarriers self-assembled from mPEG-b-PLA (“PLA”) and mPEG-b-PCL (“PCL”) were taken up more slowly by HEK293T cells (FIG. 2, B-C).

These results confirm cellular uptake of nanocarriers proceeds rapidly, within 2 h, and further suggest metabolic changes will begin to be detectable shortly after administration in future tracing studies.

### Example 3: Nanocarrier Delivery Enables the Greater Intracellular Accumulation of U-¹³C-Palmitate Tracer Compared to the Traditional BSA Strategy

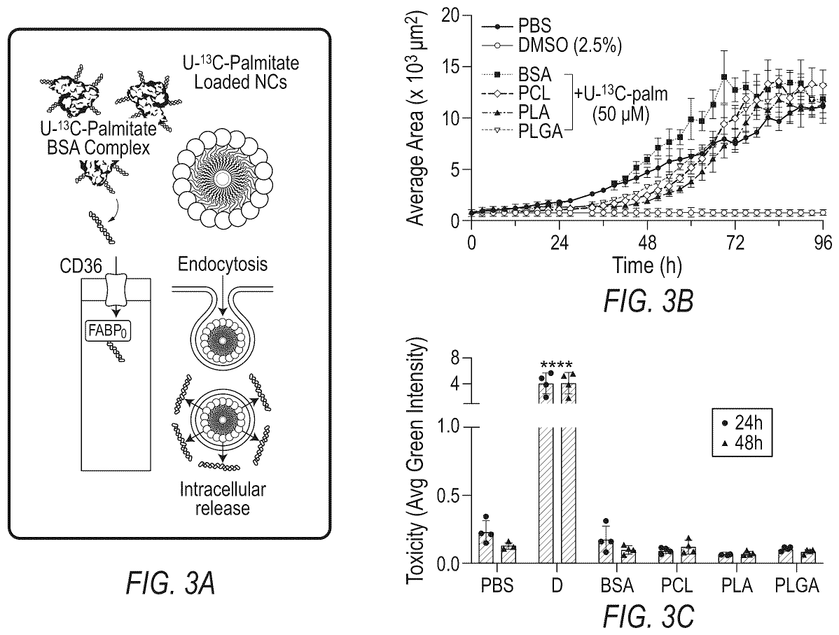

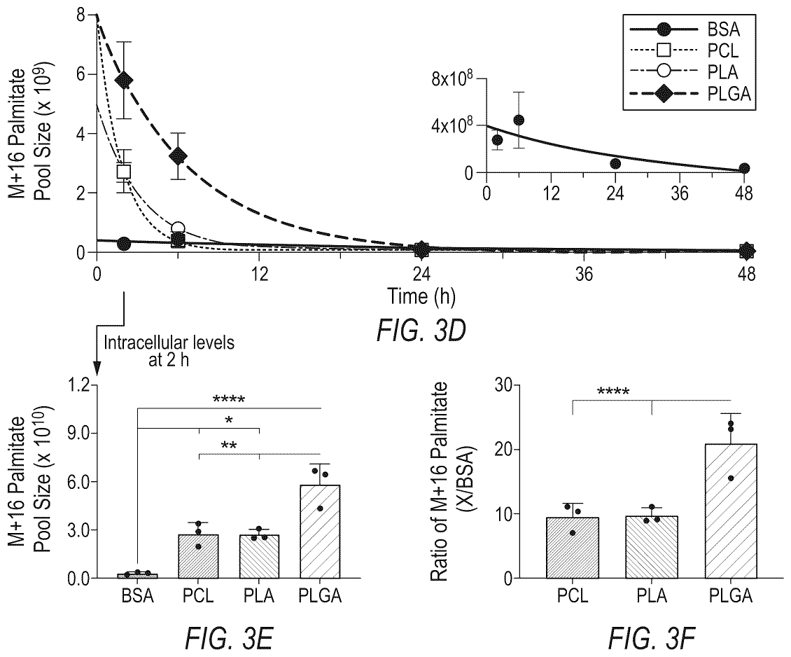

The suitability of the polymeric nanocarriers for delivering hydrophobic stable isotope labeled (SIL) substrates that are commonly used in metabolomics and lipidomics investigations was assessed in detail. U-¹³C-palmitate was prepared as either a traditional complex with bovine serum albumin (BSA) or as a nanocarrier-encapsulated form. Comparing intracellular palmitate tracer levels across conditions shortly after administration was of interest to us since the BSA method and nanocarrier methods are expected to deliver payloads using different mechanisms. The BSA-bound form solubilized palmitate tracer as a non-covalent complex and acts as a ferry that effectively transfers the cargo to transporters at the cell surface, such as CD36 (FIG. 3A). In contrast, nanocarriers encapsulate large quantities of U-¹³C-palmitate molecules within their hydrophobic core, enter cells via endocytosis, release the payload intracellularly (FIG. 3A). Considering this, it was hypothesized that the nanocarriers would deliver the hydrophobic payloads more efficiently than BSA.

Given our observation that nanocarrier uptake increased significantly from 2 h to 24 h, we included an additional 6 h timepoint in our metabolomics and lipidomics studies. Cells were treated with a U-¹³C-palmitate concentration of 50 μM, delivered via complexation with BSA or as a nanocarrier-encapsulated form, for 2 hours, 6 hours, 24 hours, or 48 hours.

Importantly, this 50 μM concentration of U-¹³C-palmitate is non-toxic to HEK293T cells (FIG. 3, B-C). Cells treated with BSA- or nanocarrier-delivered palmitate proliferated at similar rates to cells treated with PBS (FIG. 3B). Cytotoxicity differences were not observed between palmitate treatment groups and PBS through 48 h (FIG. 3C). Spent media and cells were processed using a standard 4:4:2 acetonitrile:methanol:water extraction procedure. Data was collected using an ion-paired method on an Orbitrap Exploris™ 240 mass spectrometer.

The fully labeled (M+16) species of U-¹³C-palmitate tracer gradually depleted from the media with time as cells internalized the tracer delivered using either BSA or polymeric nanocarriers (FIG. 9). Labeled palmitate (FA 16:0) was detectable in cell extracts by 2 h—the earliest timepoint examined (FIG. 3C, D). Higher levels of M+16 palmitate in the media (FIG. 9) corresponded to lower levels of this tracer in the cell extracts and vice versa (FIG. 3D). These results confirmed the tracer was successfully internalized by cells regardless of the transporter mechanism used (FIG. 3A).

The intracellular levels of labeled palmitate at 2 h were significantly greater in the nanocarrier treatment groups compared to the levels achieved when delivered as a traditional BSA-palmitate complex (FIG. 3E). Depending on the nanocarrier system used, the tracer accumulated to levels that were 10-20 times higher than the level observed in the BSA group. Palmitate accumulation was greatest in the PLGA group, which is a size-dependent phenomena. The PLGA nanocarriers are the smallest structure in this study (Table 2) and are internalized more rapidly than PCL or the closely related PLA nanocarriers of larger size (FIG. 2).

Tracer release profiles were also distinct between the nanocarrier and BSA groups. When nanocarriers were used for delivery, the intracellular palmitate rapidly depleted with a non-linear profile (FIG. 3D). A consistent, monotonic decrease in tracer was observed with time indicative of straightforward first-order depletion kinetics. In contrast, BSA-delivery yielded a slower tracer depletion profile that was linear (FIG. 3D). A subtle increase was observed at 6 h prior to depletion suggesting delayed or biphasic uptake. This difference is likely attributable to transport limitations.

Compared to the BSA delivery form, these results demonstrate that nanocarriers bypass certain limitations imposed by membrane transporters, particularly bottlenecks, to achieve greater intracellular palmitate concentrations shortly after administration. This caveat is significant because the nanocarriers produce an intracellular bolus that floods the system with the hydrophobic tracer, which is a delivery property that can be leveraged to improve metabolite labeling-likely through mass action-based considerations.

### Example 4: Lipidomics Analysis Reveals the Time-Dependent Labeling of Complex Lipids Achieved by Nanocarrier Tracer Delivery Correlates with that of the Standard Albumin-Based Approach

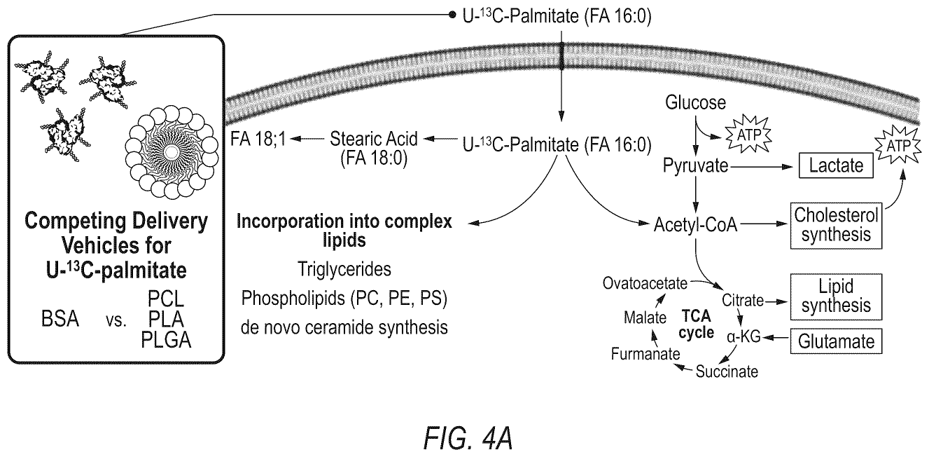

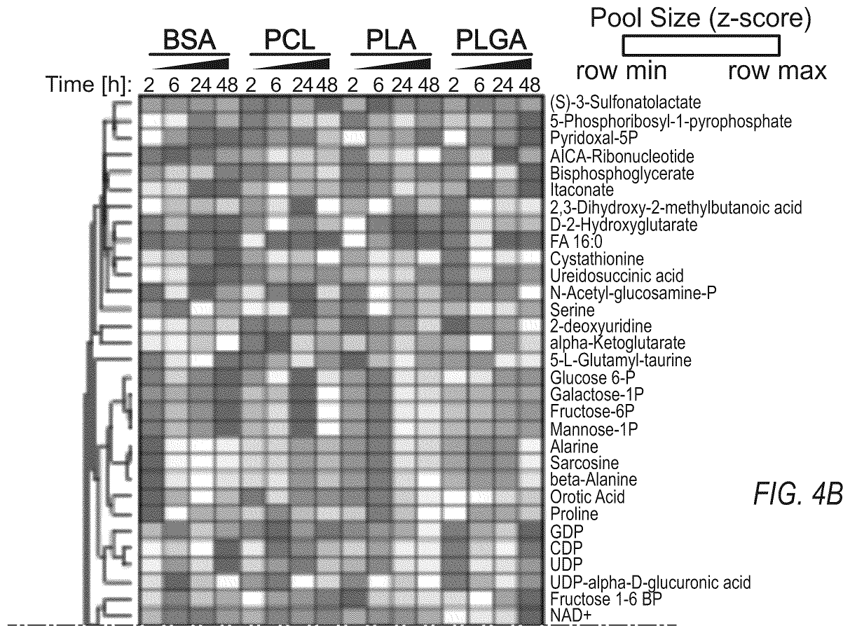

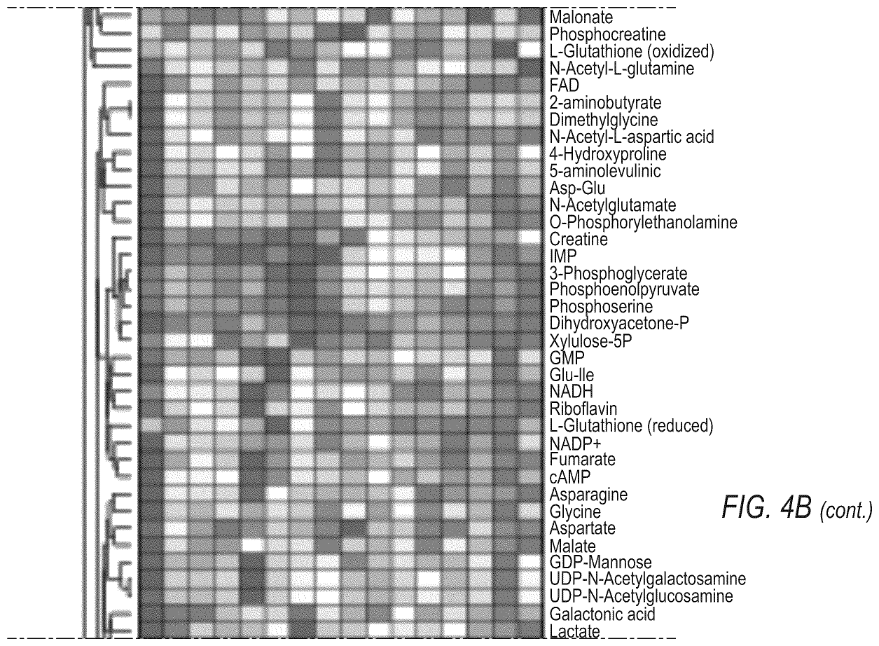

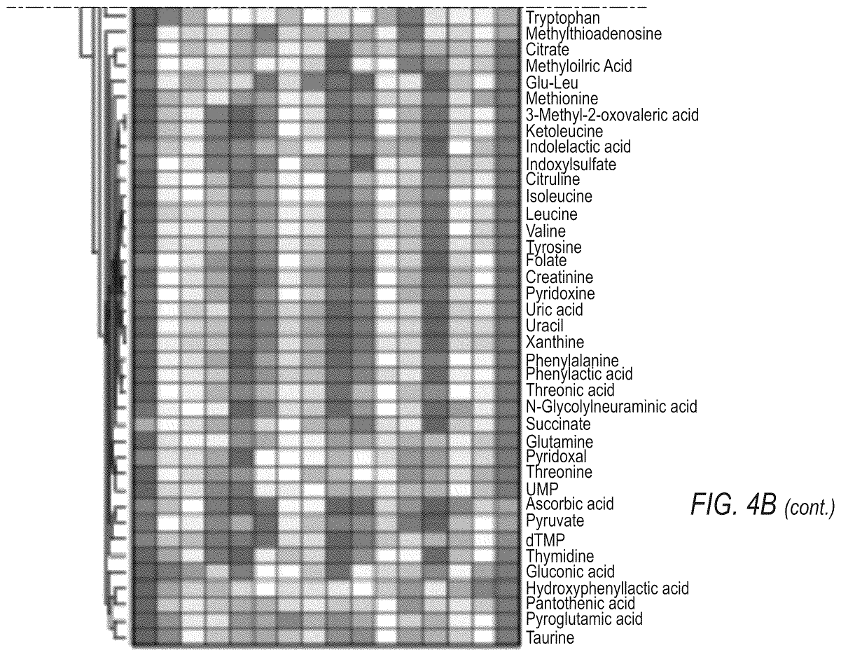

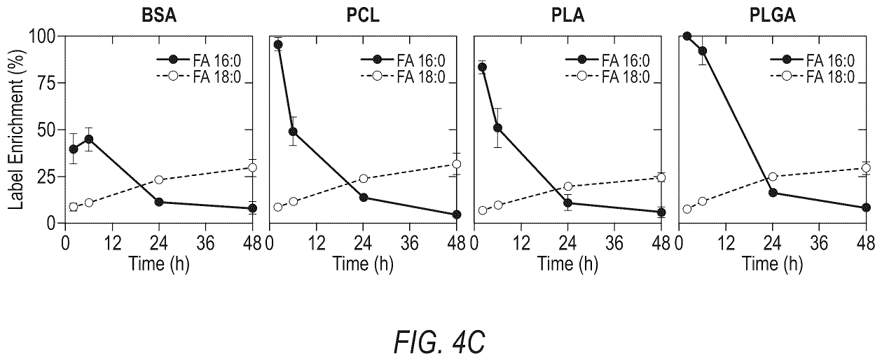

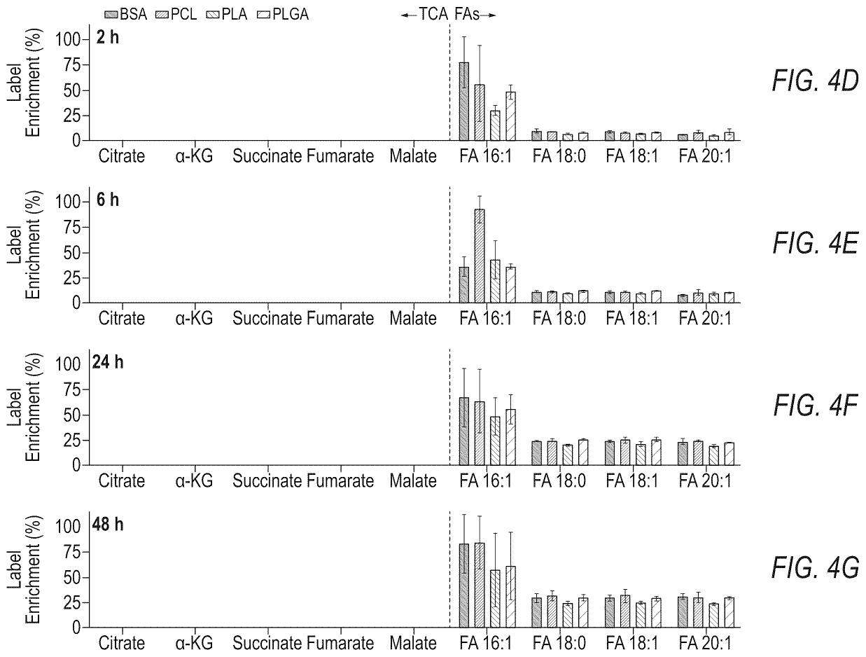

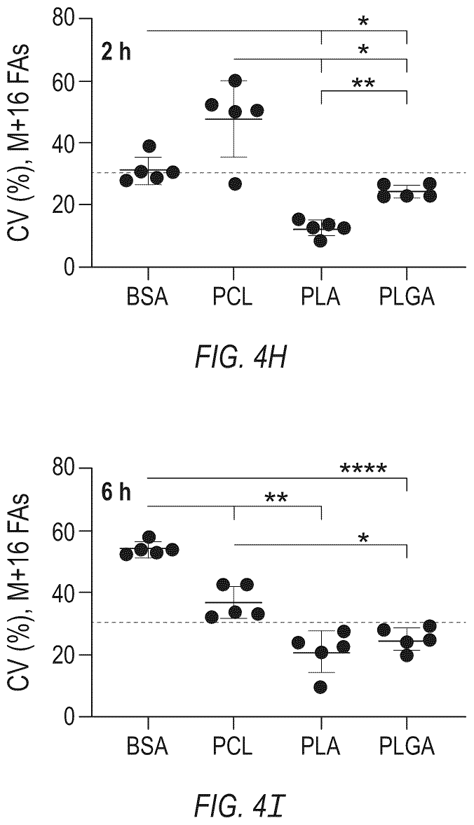

The time-dependent increases in FA labeling together with the minimal TCA labeling in the aforementioned metabolism led us to examine tracer incorporation into complex lipids. It was hypothesized that the time-dependent labeling patterns in complex lipids would be similar in cells receiving labeled palmitate delivered using nanocarriers versus the traditional BSA conjugates, but that nanocarriers would achieve more consistent labeling. Differences in labeling variation were hypothesized based on the observations for FA labeling covered earlier in this document (FIG. 4, H-I).

Complex lipids were isolated by performing Bligh-Dyer extractions on cells. Data was collected using a Orbitrap ID-X (ThermoFisher Scientific). The resulting dataset included various complex lipid species, including ceramides (Cer), diradylglycerolipids (DG), phosphatidylcholines (PC), phosphatidylethanolamines (PE), glycerophospholipids (PI), and triglycerides (TG).

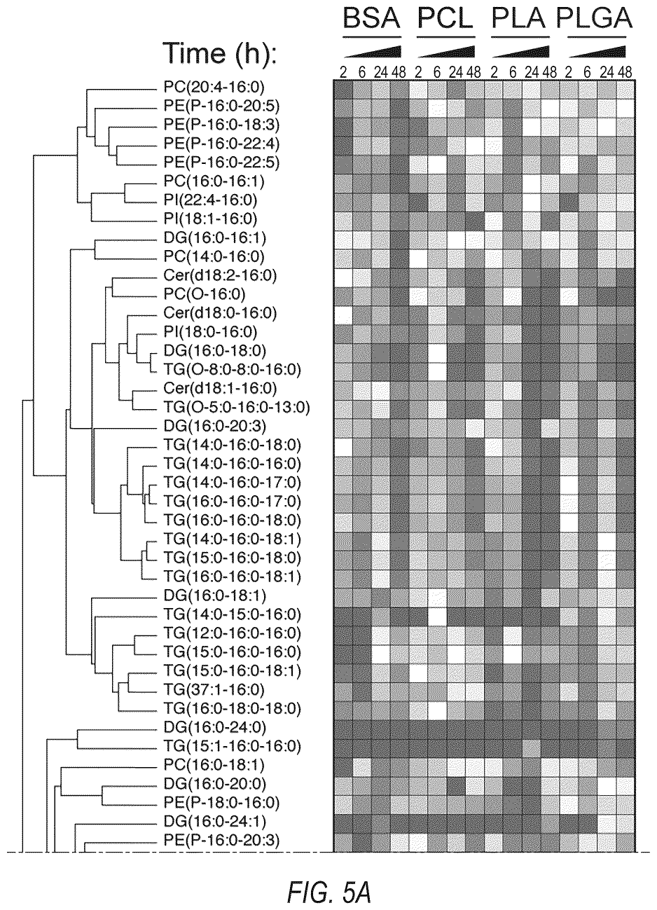

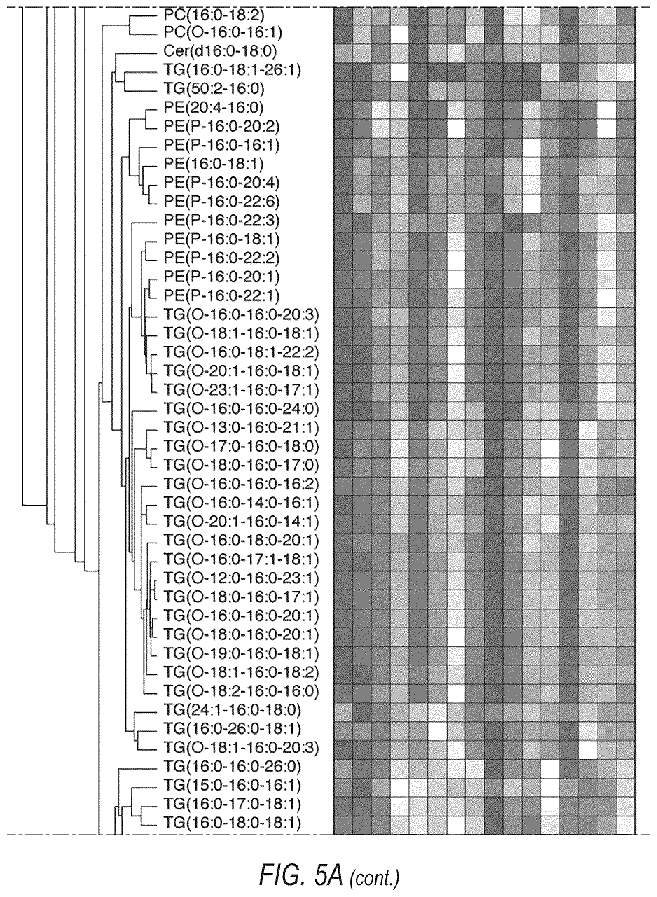

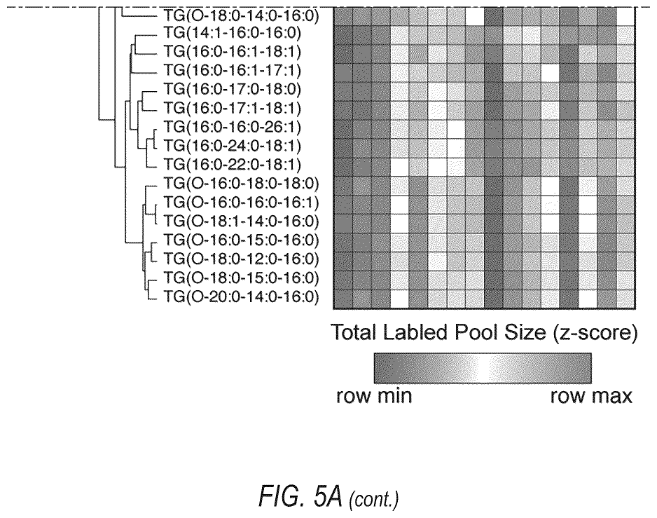

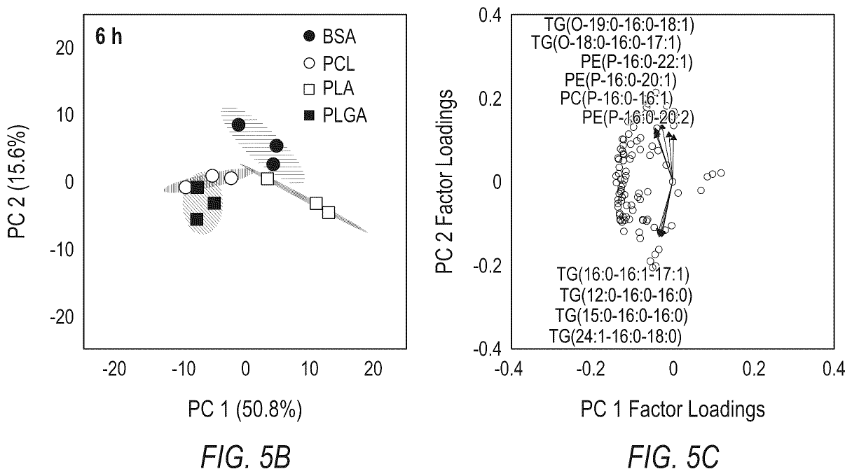

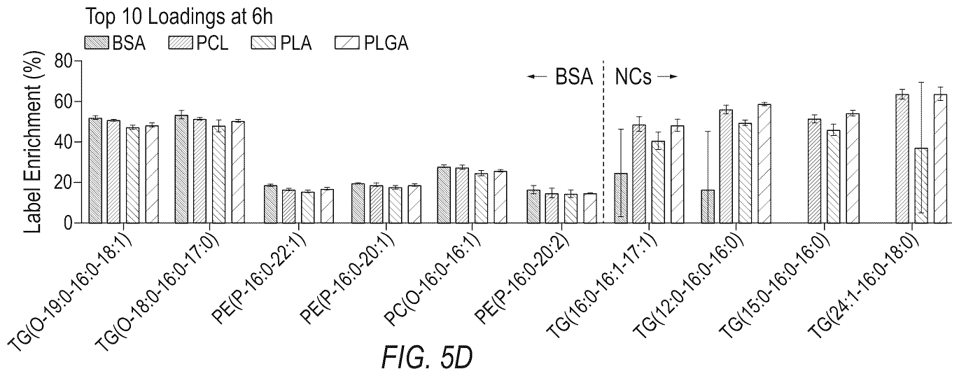

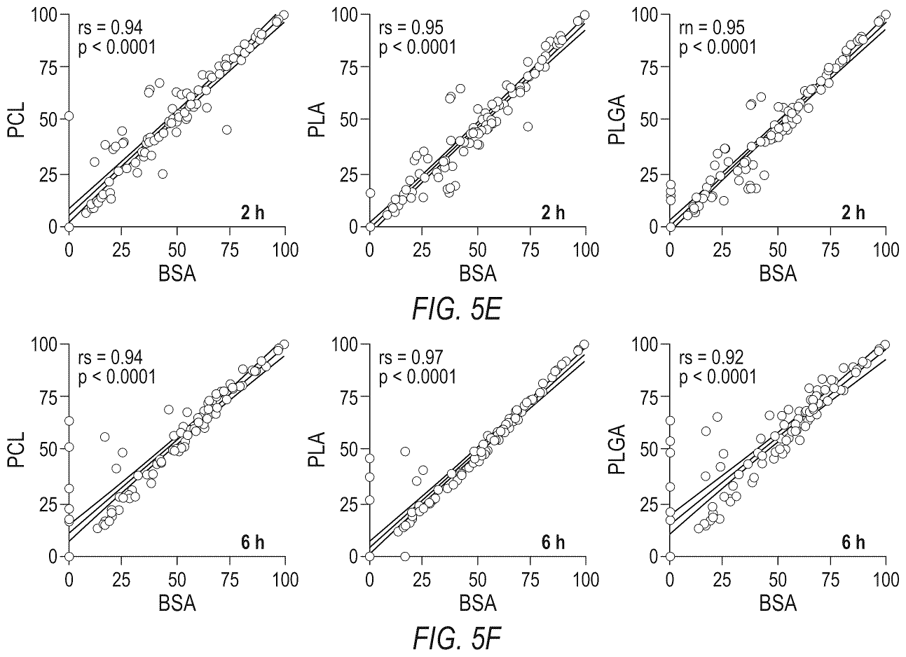

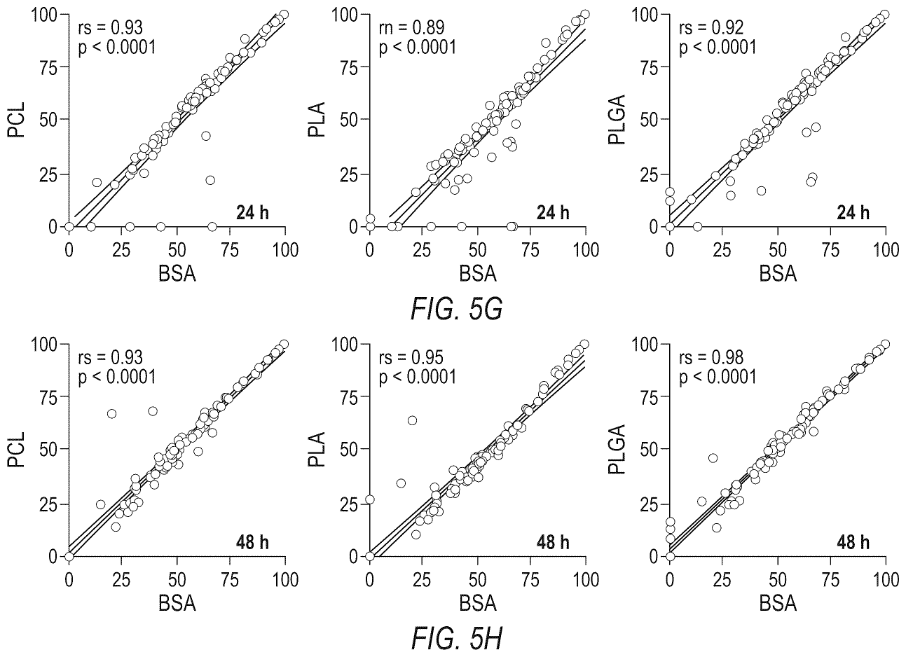

Label incorporation into various lipid species was observed (FIG. 5A). Various triglycerides, diradylglycerolipids, phosphatidylethanolamines, and glycerophospholipids were labeled at all timepoints (FIG. 5A). Closer inspection of the clades revealed a variety of time-dependent trends. For example, a subset of triglycerides including 16-carbon branches were found to be highly labeled at early 2 hour and 6 hour timepoints yet diminished with time (FIG. 5A). Ether lipids were found to have a distinct temporal trend, exhibiting low label incorporation early on but increasing gradually with time as more of the palmitate tracer was incorporated into complex lipids (FIG. 5A).

The focus of this lipidomics analysis was to understand whether nanocarrier delivery systems achieve similar labeling to that observed in samples receiving labeled palmitate in complex with BSA. This is important since a major goal of these nanocarrier development efforts is to develop a technology that captures the labeling trends of the canonical approach while achieving significant performance enhancements in terms of delivery. To this end, pairwise correlations between the nanocarrier and BSA treatment groups were examined (FIG. 5, B-M). The labeling profile of complex lipids strongly correlated between nanocarrier and BSA treatment groups at each timepoint examined, with Spearman correlation coefficients of greater than or equal to 0.89 in all cases (FIG. 5, B-M). Furthermore, each correlation was statistically significant.

While the label of labeling in these delivery groups was similar for the majority of lipids, a handful of time-dependent differences were observed as well. There were a variety of cases at 6 h and 48 h in which high labeling of certain lipid species was found in the nanocarrier groups but not the BSA delivery group (FIG. 5, E-H).

Collectively, the results from our lipidomics analysis demonstrate strong agreement in complex lipid labeling profiles between the nanocarrier and BSA delivery groups. These results demonstrate that nanocarriers capture the trends identified by the canonical BSA-based tracer delivery approach. Thus, nanocarriers provide suitable vehicles for complex lipid tracing applications and have several properties that improve delivery performance over canonical approaches. These benefits are covered elsewhere in the present document.

The results demonstrate the present nanocarriers are versatile delivery vehicles for hydrophobic tracers in metabolomics and lipidomics studies. The present disclosure captures the labeling trends obtained using canonical methods while providing several key benefits that are presently unmatched by existing technologies used in metabolic labeling studies. The nanocarriers provide safe elevations in intracellular concentrations of stable isotope labeled hydrophobic compounds, such as U-¹³C-palmitate. Higher intracellular tracing levels produce labeling with less variation/noise than the canonical methods. Furthermore, the nanocarriers are highly customizable. The physical and chemical properties of these nanocarriers can be tuned for a variety of purposes, including but not limited to, tailoring the cell selectivity of tracer uptake and the release rate of the tracer within cells.

### Example 5: Methods

Nanocarrier formulation and physicochemical characterization. Nanocarriers were prepared from mPEG2k-b-PCL10k, mPEG2k-b-PLA10k, and mPEG2k-b-PLGA10k polymers (Table 1) by method of nanoprecipitation using a confined impingement jets mixer following established procedures. Briefly, polymer amphiphiles and specified hydrophobic cargo dissolved in tetrahydrofuran solvent were impinged against a stream of phosphate buffered saline (PBS) into a 1.5 mL PBS reservoir and were incubated in a desiccator overnight to remove any residual organic solvent. For tracing studies, potassium U-¹³C-palmitate (Cambridge Isotope Laboratories, CLM-3943) was dissolved in DMSO and integrated into self-assembled polymeric nanocarriers using established methods. Formulations were purified using an LH-20 column. Nanocarrier size and charge was characterized by dynamic light scattering (DLS) and electrophoretic light scattering (ELS), respectively, using a Zetasizer Nano-ZS (Malvern Panalytical, Ltd). The number-average diameter and polydispersity index (PDI), as well as the zeta potential were characterized.

Albumin-palmitate preparation. Fatty acid-free bovine serum albumin (BSA; Sigma, A8806) was used to formulate albumin-palmitate complexes.

Transmission electron microscopy (TEM). A 10 μL volume of the specified nanocarrier formulation was deposited onto a Formvar carbon-coated copper grid (Electron Microscopy Sciences) and was incubated at room temperature for 1-2 min to allow for specimen adsorption. The suspension was gently removed with filter paper. Specimens were negatively stained by applying a 10 μL volume of Uranyless heavy metal stain followed by a one-minute incubation period at room temperature. The grid was subsequently blotted with filter paper to removed excess stain. High magnification images were collected using a Tecnai Spirit transmission electron microscope operating at 120 kV ImageJ software was used for basic image processing.

Cell culture. Human embryonic kidney 293T (HEK293T) cells were obtained as a generous gift from Russell Jones' laboratory (Van Andel Institute). Unless otherwise indicated, cells were cultured at 37° C., 5% CO₂ in Dulbecco's Modified Eagle Medium (DMEM; Wisent Inc., Cat No. 319-015-CL) containing 4.5 g/L glucose, L-glutamine, 1× penicillin and streptomycin antibiotics (Gibco), and 10% fetal bovine serum (FBS). The media lacked sodium pyruvate.

Cell viability assays. HEK293T cells were seeded at 10,000 cells/well in clear, flat bottom 96-well plates at 37° C., 5% CO₂ and were allowed to adhere overnight prior to the introduction of test agents. For nanocarrier toxicity assays, cargo-free nanocarriers prepared in PBS were dosed at a working (in well) polymer concentration of 1 mg/mL. For BSA-palmitate toxicity assays, cells were treated with a working (in well) palmitate concentration of 1 μM, 10 μM, or 100 μM. In all cases, cells were incubated with test agent for a period of 24 hours. In all cases, sterile PBS was included as a non-toxic vehicle control whereas incubation with 10% DMSO was included as a control for toxicity. The MTT labeling reagent was prepared by dissolving thiazolyl blue tetrazolium bromide (Sigma, M5655) in sterile phosphate-buffered saline (PBS) at a stock concentration of 5 mg/mL. Cells were treated with MTT labeling reagent at a working concentration of 0.5 mg/mL and were incubated at 37° C., 5% CO₂ for an additional 3-4 hours until a purple precipitate (formazan crystals) were observed. The formazan crystals were solubilized in DMSO. The absorbance of 570 nm light was measured using a BioTek Synergy Neo2 multi-mode microplate reader. Cell viability was calculated as a percentage via Eq. 1:

$$Cell viability = \frac{{A}_{sample}}{{A}_{control}} * 100 \qquad Eq . 1$$

The mean cell viability±s.d. (n=3) was determined. Significant differences in cell viability were determined with one-way ANOVA with post hoc Tukey's multiple comparisons test and a 5% significance level.

Assessment of nanocarrier uptake in vitro. The cellular uptake of nanocarriers loaded with DiI dye (λ_{EX}=549 nm; λ_{Em}=565 nm) was performed using established procedures. HEK293T cells were seeded in 24 well plates at a density of 100,000 cells per well, and were cultured at 37° C., 5% CO₂ using the aforementioned media conditions (see “Cell culture” methods section). Cells were allowed to adhere overnight prior to beginning treatments. Cells were treated with DiI-loaded mPEG2k-b-PCL10k, mPEG2k-b-PLA10k, and mPEG2k-b-PLGA10k nanocarriers at an in-well polymer concentration of 0.1 mg/mL, or an equal volume of PBS (negative control) at 48 h, 24 h, or 2 h timepoints prior to harvesting for flow cytometry. Untreated cells were included as an additional control group. All conditions included three biological replicates per treatment group (n=3). At the time of harvesting, media was aspirated, and cells were washed with sterile PBS. Cells were isolated, stained with Zombie Aqua viability stain (λ_{Ex}=380 nm; λ_{Em}=512 nm; BioLegend) in cell-staining buffer (BioLegend), and fixed with a paraformaldehyde-based fixation buffer (BioLegend). Dead cell controls were prepared for viability staining by incubating cells at 85° C. (heat treatment) for 5 min, allowing the heated cell suspension to cool, and mixing with live cells (FIG. 7A). Unstained and single-color compensation controls were prepared for use in flow cytometry setup and analysis. Flow cytometry was performed using a Cytek Aurora spectral flow cytometer. Data was analyzed using Cytobank software. DiI fluorescence was used to quantify nanocarrier uptake in live single cells. The percentage of nanocarrier positive cells (% NC+ cells) was quantified with a false positive rate of <1% (FIG. 7B).

Cell proliferation and cytotoxicity assays. Flat-bottom 96-well plates (Fisher, FB012931) were seeded with 5,000 cells per well in complete DMEM supplemented with 20 nM SYTOX green nucleic acid stain (Invitrogen, S7020). Cells were treated with BSA or nanocarrier formulations at the specified material or U-¹³C-palmitate concentrations. Cellular proliferation and SYTOX staining was monitored using an Incucyte (Sartorius) live cell imaging system. Phase contrast and green channel images were acquired at 20× magnification. The average area (μm²) and average mean green intensity were analyzed.

Palmitate tracing studies in vitro. HEK293T cells were seeded in 6 well plates at a density of 100,000 cells per well, cultured at 37° C., 5% CO₂ using the aforementioned media conditions (see “Cell culture” methods section), and were allowed to adhere overnight prior to beginning treatments. Cells were treated with U-¹³C-palmitate either complexed with albumin or loaded into mPEG2k-b-PCL10k, mPEG2k-b-PLA10k, and mPEG2k-b-PLGA10k nanocarriers at an in-well U-¹³C-palmitate concentration of 50 μM for 48 h, 24 h, 6 h, or 2 h prior to harvesting. At harvest, media was aspirated, cells were washed with a cold NaCl solution and were stored at −80° C. before performing extraction procedures.

Metabolomics. Small molecule metabolites, including TCA intermediates and fatty acids, were extracted by subjecting cells or media to cold acetonitrile (ACN; Fisher, A955-4), methanol (MeOH; Fisher, A456-5) and water (Fisher, W6-4) in a 4:4:2 (v/v/v) ratio. Samples were vortexed for 10 sec, sonicated for 5 min, and were incubated on wet ice for 1 hour. Samples were centrifuged subsequently for 10 min at 17,000×g, 4° C. The supernatants were collected and were subjected to a second round of centrifugation at 17,000×g, 4° C. for 10 min to ensure the complete precipitate removal. A volume of 800 μL of each supernatant was collected and dried down via rotary evaporation. Samples were resuspended in 50 μL of cold water and ACN mixed at a 1:1 ratio. Samples were vortexed, sonicated, and centrifuged at 17,000×g, 4° C. prior to transferring the supernatant to autosampler vials.

For metabolomics data acquisition, samples were analyzed with a Thermo Vanquish dual liquid chromatography system utilizing two alternating methods coupled to an Orbitrap Exploris 240 (Thermo Fisher Scientific) using an H-ESI (heated electrospray ionization) source operating in negative mode. The two alternating methods used the same chromatography and mass spectrometer settings. A 2 μL volume of each sample was injected and run through a reversed-phase ZORBAX RRHD Extend-C18 Column (1.8 m; 2.1 mm×150 mm; Agilent, 759700-902) combined with a UHPLC guard column (1.8 m; 2.1 mm×5 mm; Agilent, 821725-907) over a period of 24 min. Mobile phase A consisted of LC/MS grade water (Fisher, W6) with 3% LC/MS grade methanol (Fisher, A456), mobile phase B was LC/MS grade methanol and both mobile phases contained 10 mM tributylamine (Sigma, 90780), 15 mM LC/MS grade acetic acid (Fisher, A11350), and 0.01% medronic acid (v/v; Agilent, 5191-4506). For the wash gradient, mobile phase A was kept the same, and mobile phase B was 99% LC/MS grade acetonitrile (Fisher, A955). The temperature of the column was set to 35° C. The solvent flow rate was held at 0.25 mL/min. The chromatography gradient was as follows: 0-2.5 min held at 0% B, 2.5-7.5 min from 0% B to 20% B, 7.5-13 min from 20% B to 45% B, 13-20 min from 45% B to 99% B, and 20-24 min held at 99% B. A 24 minute wash gradient was run in between every injection (in parallel with the alternating method chromatography) in reverse flow direction to back-flush the column and to re-equilibrate solvent conditions as follows: 0-1.6 min held at 99% B and 0.25 mL/min, 1.6-2.4 min held at 99% B and ramp to 0.8 mL/min, 2.4-13.95 min held at 99% B and 0.8 mL/min, 13.95-14.1 min held at 99% B and ramp to 0.6 mL/min, 14.1-14.85 min from 99% B to 0% B and ramp to 0.4 mL/min, 14.85-22.5 min held at 0% B and 0.4 mL/min, 22.5-22.6 min held at 0% B and ramp to 0.25 mL/min, and 22.6-24 min held at 0% B and 0.25 mL/min. The mass spectrometer parameters were as follows: source voltage −2500 V, sheath gas 60, aux gas 19, sweep gas 1, ion transfer tube temperature 320° C., and vaporizer temperature 250° C. Full scan data were collected using the orbitrap with a scan range of 70-850 m/z at a resolution of 240,000 and RF lens at 35%. Fragmentation was induced in the orbitrap using assisted higher-energy collisional dissociation (HCD) collision energies at 15, 30, and 45%. The orbitrap resolution was 15,000, the isolation window was 2 m/z, and the data dependent scans were capped at 5 scans. Targeted mass MS2 triggers were included for a panel of compounds. These compounds are presented in Table 3.

TABLE 3
Summary of Targeted Mass MS2 Triggers for Metabolites Analyzed Using the Ion-Paired Method.

| | | | | | RT | Window |
|---|---|---|---|---|---|---|
| Compound | Formula | Adduct | m/z | Z | (min) | (min) |
| 2,3-Dihydroxy-2-methylbutanoic acid | C5H10O4 | —H | 133.0506 | 1 | 8.78 | 2 |
| D-2-Aminobutyrate | C4H9NO2 | —H | 102.0561 | 1 | 1.2 | 2 |
| Deoxyuridine | C9H12N2O5 | —H | 227.0673 | 1 | 2.1 | 2 |
| 2-Hydroxybutyrate | C4H8O3 | —H | 103.0401 | 1 | 9.36 | 2 |
| 3-D-Hydroxybutyrate | C4H8O3 | —H | 103.0401 | 1 | 7.57 | 2 |
| 3-Hydroxyisobutyrate | C4H8O3 | —H | 103.0401 | 1 | 7.36 | 2 |
| 3-Methyl-2-oxovaleric acid | C6H10O3 | —H | 129.0557 | 1 | 15.58 | 2 |
| 3-Phosphoglyceric acid | C3H7O7P | —H | 184.9857 | 1 | 13.75 | 2 |
| 4-Hydroxybutyrate | C4H8O3 | —H | 103.0401 | 1 | 8.65 | 2 |
| 4-Hydroxyproline | C5H9NO3 | —H | 130.051 | 1 | 1.16 | 2 |
| 5-Aminolevulinic acid | C5H9NO3 | —H | 130.051 | 1 | 1.15 | 2 |
| Phosphoribosyl pyrophosphate | C5H13O14P3 | —H | 388.9445 | 1 | 16.11 | 2 |
| Adenine | C5H5N5 | —H | 134.0472 | 1 | 2.5 | 3 |
| Adenosine | C10H13N5O4 | —H | 266.0895 | 1 | 5.6 | 2 |
| Adenosine 3-phosphate 5-phosphosulfate | C10H15N5O13P2S | —H | 505.979 | 1 | 16.26 | 2 |
| ADP-Ribose | C15H23N5O14P2 | —H | 558.0644 | 1 | 13.94 | 2 |
| CAMP | C10H12N5O6P | —H | 328.0452 | 1 | 12.49 | 2 |
| ADP | C10H15N5O10P2 | —H | 426.0221 | 1 | 14.34 | 2 |
| AICAR | C9H15N4O8P | —H | 337.0555 | 1 | 9.86 | 2 |
| Alanine | C3H7NO2 | —H | 88.0404 | 1 | 1.18 | 2 |
| alpha-Ketoglutarate | C5H6O5 | —H | 145.0142 | 1 | 13.31 | 2 |
| AMP | C10H14N5O7P | —H | 346.0558 | 1 | 11.11 | 2 |
| Arachidonic acid | C20H32O2 | —H | 303.233 | 1 | 20.054 | 2 |
| Ascorbic acid | C6H8O6 | —H | 175.0248 | 1 | 14.16 | 2 |
| Asparagine | C4H8N2O3 | —H | 131.0462 | 1 | 1.15 | 2 |
| Aspartate | C4H7NO4 | —H | 132.0302 | 1 | 3.99 | 2 |
| Asp-Asp | C8H12N2O7 | —H | 247.0572 | 1 | 12.388 | 2 |
| Asp-Glu | C9H14N2O7 | —H | 261.0728 | 1 | 12.3 | 2 |
| Asp-Gly | C6H10N2O5 | —H | 189.0517 | 1 | 3.741 | 2 |
| ATP | C10H16N5O13P3 | —H | 505.9885 | 1 | 16.2 | 2 |
| Bilirubin | C33H36N4O6 | —H | 583.2562 | 1 | 21.71 | 2 |
| Bisphosphoglycerate | C3H8O10P2 | —H | 264.952 | 1 | 16.18 | 2 |
| CDP | C9H15N3O11P2 | —H | 402.0109 | 1 | 13.48 | 2 |
| CDP-ethanolamine | C11H20N4O11P2 | —H | 445.0531 | 1 | 5.4 | 2 |
| CDP-n-acetylneuraminic acid | C20H31N4O16P | —H | 613.14 | 1 | 13.309 | 2 |
| Citraconic acid | C5H6O4 | —H | 129.0193 | 1 | 13.3 | 2 |
| Citrate | C6H8O7 | —H | 191.0197 | 1 | 13.92 | 2 |
| Citrulline | C6H13N3O3 | —H | 174.0884 | 1 | 1.18 | 2 |
| CMP | C9H14N3O8P | —H | 322.0446 | 1 | 8.64 | 2 |
| Coenzyme A | C21H36N7O16P3S | —H | 766.1079 | 1 | 16.9 | 2 |
| Creatine | C4H9N3O2 | —H | 130.0622 | 1 | 1.2 | 2 |
| Creatinine | C4H7N3O | —H | 112.0516 | 1 | 1.29 | 2 |
| CTP | C9H16N3O14P3 | —H | 481.9772 | 1 | 15.92 | 2 |
| Cystathionine | C7H14N2O4S | —H | 221.0602 | 1 | 1.15 | 2 |
| Cytidine | C9H13N3O5 | —H | 242.0782 | 1 | 1.62 | 2 |
| Cytosine | C4H5N3O | —H | 110.036 | 1 | 1.15 | 2 |
| D-2-Hydroxyglutaric acid | C5H8O5 | —H | 147.0299 | 1 | 12.85 | 2 |
| dCMP | C9H14N3O7P | —H | 306.0497 | 1 | 9.635 | 2 |
| Dihydroorotic acid | C5H6N2O4 | —H | 157.0255 | 1 | 6.33 | 2 |
| Dihydroxyacetone phosphate | C3H7O6P | —H | 168.9907 | 1 | 9.39 | 2 |
| Dimethylglycine | C4H9NO2 | —H | 102.0561 | 1 | 1.15 | 2 |
| Dopamine | C8H11NO2 | —H | 152.0717 | 1 | 1.08 | 2 |
| dTDP | C10H16N2O11P2 | —H | 401.0157 | 1 | 14.38 | 2 |
| dTMP | C10H15N2O8P | —H | 321.0493 | 1 | 10.91 | 2 |
| dTTP | C10H17N2O14P3 | —H | 480.982 | 1 | 16.26 | 2 |
| FA 16:0 | C16H32O2 | —H | 255.233 | 1 | 22.34 | 3 |
| FA 16:1 | C16H30O2 | —H | 253.2173 | 1 | 21.91 | 2 |
| FA 18:0 | C18H36O2 | —H | 283.2643 | 1 | 23.07 | 1.5 |
| FA 18:1 | C18H34O2 | —H | 281.2486 | 1 | 22.44 | 2 |
| FA 18:2 | C18H32O2 | —H | 279.233 | 1 | 22.05 | 2 |
| FA 18:3 | C18H30O2 | —H | 277.2173 | 1 | 21.77 | 2 |
| FA 20:1 | C20H38O2 | —H | 309.2799 | 1 | 23.14 | 1 |
| FA 20:2 | C20H36O2 | —H | 307.2643 | 1 | 22.65 | 2 |
| FA 20:3 | C20H34O2 | —H | 305.2486 | 1 | 22.23 | 3 |
| FA 22:2 | C22H40O2 | —H | 335.2956 | 1 | 23.34 | 1 |
| FA 22:5 | C22H34O2 | —H | 329.2486 | 1 | 21.99 | 2 |
| FA 22:6 | C22H32O2 | —H | 327.233 | 1 | 21.91 | 2 |
| Farnesyl pyrophosphate | C15H28O7P2 | —H | 381.1237 | 1 | 20.64 | 2 |
| FAD | C27H33N9O15P2 | —H | 784.1499 | 1 | 16.05 | 2 |
| FMN | C17H21N4O9P | —H | 455.0973 | 1 | 14.81 | 2 |
| Folic acid | C19H19N7O6 | —H | 440.1324 | 1 | 14.23 | 2 |
| Fructose 1,6-BP | C6H14O12P2 | —H | 338.9888 | 1 | 14.004 | 2 |
| Fumaric acid | C4H4O4 | —H | 115.0037 | 1 | 13.7 | 2 |
| GABA | C4H9NO2 | —H | 102.0561 | 1 | 1.17 | 2 |
| Galactonic acid | C6H12O7 | —H | 195.051 | 1 | 4.58 | 2 |
| gamma-Glu-Cys | C8H14N2O5S | —H | 249.0551 | 1 | 7.486 | 2 |
| GDP | C10H15N5O11P2 | —H | 442.0171 | 1 | 13.83 | 2 |
| GDP-Mannose | C16H25N5O16P2 | —H | 604.0699 | 1 | 13.25 | 2 |
| Gln-Glu | C10H17N3O6 | —H | 274.1045 | 1 | 5.62 | 2 |
| Glu-Ala | C8H14N2O5 | —H | 217.083 | 1 | 7.534 | 2 |
| Gluconic acid | C6H12O7 | —H | 195.051 | 1 | 4.84 | 2 |
| Glu-Gly | C7H12N2O5 | —H | 203.0673 | 1 | 4.672 | 2 |
| Glu-Ile | C11H20N2O5 | —H | 259.1299 | 1 | 12.365 | 2 |
| Glutaconic acid | C5H6O4 | —H | 129.0193 | 1 | 13.69 | 2 |
| Glutamate | C5H9NO4 | —H | 146.0459 | 1 | 3.9 | 2 |
| Glutamine | C5H10N2O3 | —H | 145.0619 | 1 | 1.15 | 2 |
| 5-L-Glutamyl-taurine | C7H14N2O6S | —H | 253.05 | 1 | 5.291 | 2 |
| gamma-Glu-Thr | C9H16N2O6 | —H | 247.0936 | 1 | 5.672 | 2 |
| gamma-Glu-Val | C10H18N2O5 | —H | 245.1143 | 1 | 9.995 | 2 |
| Glycerol 3-phosphate | C3H9O6P | —H | 171.0064 | 1 | 7.4 | 2 |
| Glycine | C2H5NO2 | —H | 74.0248 | 1 | 1.13 | 2 |
| GMP | C10H14N5O8P | —H | 362.0507 | 1 | 10.03 | 2 |
| GTP | C10H16N5O14P3 | —H | 521.9834 | 1 | 16.01 | 2 |
| Hexose phosphate I | C6H13O9P | —H | 259.0224 | 1 | 6.39 | 2 |
| Hexose phosphate II | C6H13O9P | —H | 259.0224 | 1 | 6.6 | 2 |
| Hexose phosphate III | C6H13O9P | —H | 259.0224 | 1 | 6.86 | 2 |
| Hexose phosphate IV | C6H13O9P | —H | 259.0224 | 1 | 7.06 | 2 |
| Hexose phosphate IX | C6H13O9P | —H | 259.0224 | 1 | 9.4 | 2 |
| Hexose phosphate V | C6H13O9P | —H | 259.0224 | 1 | 7.19 | 2 |
| Hexose phosphate VI | C6H13O9P | —H | 259.0224 | 1 | 7.41 | 2 |
| Hexose phosphate VII | C6H13O9P | —H | 259.0224 | 1 | 8.6 | 2 |
| Hexose phosphate VIII | C6H13O9P | —H | 259.0224 | 1 | 9.16 | 2 |
| Hexose phosphate X | C6H13O9P | —H | 259.0224 | 1 | 10.49 | 2 |
| Histidine | C6H9N3O2 | —H | 154.0622 | 1 | 1.04 | 2 |
| Hydroxydecanoic acid | C10H20O3 | —H | 187.134 | 1 | 17.32 | 2 |
| Hydroxyhexadecanoic acid | C16H32O3 | —H | 271.2279 | 1 | 21.58 | 2 |
| 5-Hydroxyindoleacetic acid | C10H9NO3 | —H | 190.051 | 1 | 12.09 | 2 |
| 2-Hydroxystearic acid | C18H36O3 | —H | 299.2592 | 1 | 22.037 | 3 |
| Hypoxanthine | C5H4N4O | —H | 135.0312 | 1 | 1.7 | 2 |
| IMP | C10H13N4O8P | —H | 347.0398 | 1 | 10.01 | 2 |
| Indolelactic acid | C11H11NO3 | —H | 204.0666 | 1 | 15.53 | 2 |
| Indoxyl sulfate | C8H7NO4S | —H | 212.0023 | 1 | 15.5 | 2 |
| Inosine | C10H12N4O5 | —H | 267.0735 | 1 | 2.5 | 2 |
| Isoleucine | C6H13NO2 | —H | 130.0874 | 1 | 1.87 | 3 |
| Itaconate | C5H6O4 | —H | 129.0193 | 1 | 12.6 | 2 |
| Ketoisovalerate | C5H8O3 | —H | 115.0401 | 1 | 13.84 | 2 |
| Ketoleucine | C6H10O3 | —H | 129.0557 | 1 | 15.92 | 2 |
| Lactate | C3H6O3 | —H | 89.0244 | 1 | 6.918 | 2 |
| Leucine | C6H13NO2 | —H | 130.0874 | 1 | 2 | 3 |
| Glutathione (reduced) | C10H17N3O6S | —H | 306.0765 | 1 | 7.61 | 2 |
| Glutathione (oxidized) | C20H32N6O12S2 | —H | 611.1447 | 1 | 12.57 | 2 |
| Malate | C4H6O5 | —H | 133.0142 | 1 | 12.72 | 2 |
| Malonic acid | C3H4O4 | —H | 103.0037 | 1 | 11.72 | 2 |
| Methionine | C5H11NO2S | —H | 148.0438 | 1 | 1.6 | 2 |
| 2-Methylcitrate | C7H10O7 | —H | 205.0354 | 1 | 14.38 | 2 |
| 5′-Methylthioadenosine | C11H15N5O3S | —H | 296.0823 | 1 | 10.88 | 2 |
| N-Acetyl-D-galactosamine 1-phosphate | C8H16NO9P | —H | 300.049 | 1 | 7.92 | 2 |
| N-Acetyl-1-aspartylglutamic acid | C11H16N2O8 | —H | 303.0834 | 1 | 15.255 | 2 |
| N-Acetyl-L-alanine | C5H9NO3 | —H | 130.051 | 1 | 8.17 | 2 |
| N-Acetyl-D-Glucosamine 6-Phosphate | C8H16NO9P | —H | 300.049 | 1 | 8.198 | 2 |
| N-Acetyl-L-glutamic acid | C7H11NO5 | —H | 188.0564 | 1 | 13.15 | 2 |
| N-Acetyl-L-aspartic acid | C6H9NO5 | —H | 174.0408 | 1 | 13.05 | 2 |
| N-Acetylglutamine | C7H12N2O4 | —H | 187.0724 | 1 | 6.859 | 2 |
| N-Acetyl-L-methionine | C7H13NO3S | —H | 190.0543 | 1 | 13.1 | 2 |
| N-Acetylneuraminic acid | C11H19NO9 | —H | 308.0987 | 1 | 5.717 | 2 |
| N-Acetyl-L-phenylalanine | C11H13NO3 | —H | 206.0823 | 1 | 15.61 | 2 |
| N-Acetylserine | C5H9NO4 | —H | 146.0459 | 1 | 5.9 | 2 |
| N-Acetyltryptophan | C13H14N2O3 | —H | 245.0932 | 1 | 15.56 | 2 |
| NAD+ | C21H27N7O14P2 | —H | 662.1018 | 1 | 8.61 | 2 |
| NADH | C21H29N7O14P2 | —H | 664.1175 | 1 | 14.41 | 2 |
| NADP+ | C21H28N7O17P3 | —H | 742.0682 | 1 | 13.81 | 2 |
| NADPH | C21H30N7O17P3 | —H | 744.0838 | 1 | 16.18 | 2 |
| Carglumic acid | C6H10N2O5 | —H | 189.0517 | 1 | 12.61 | 2 |
| N-Glycolylneuraminic acid | C11H19NO10 | —H | 324.0936 | 1 | 4.95 | 2 |
| O-Acetylserine | C5H9NO4 | —H | 146.0459 | 1 | 6.37 | 2 |
| O-Phosphoethanolamine | C2H8NO4P | —H | 140.0118 | 1 | 1.65 | 3 |
| Ophthalmate | C11H19N3O6 | —H | 288.1201 | 1 | 7.85 | 2 |
| Orotic acid | C5H4N2O4 | —H | 155.0098 | 1 | 8.15 | 2 |
| Orotidine | C10H12N2O8 | —H | 287.0521 | 1 | 7.221 | 2 |
| Pantothenic acid | C9H17NO5 | —H | 218.1034 | 1 | 11.17 | 2 |
| Pentose phosphate I | C5H11O8P | —H | 229.0119 | 1 | 6.82 | 2 |
| Pentose phosphate III | C5H11O8P | —H | 229.0119 | 1 | 8 | 2 |
| Pentose phosphate IV | C5H11O8P | —H | 229.0119 | 1 | 8.7 | 2 |
| Pentose phosphate V | C5H11O8P | —H | 229.0119 | 1 | 10.33 | 2 |
| Pentose phosphate II | C5H11O8P | —H | 229.0119 | 1 | 7.8 | 2 |
| Phenylalanine | C9H11NO2 | —H | 164.0717 | 1 | 3.75 | 2 |
| Phenyllactic acid | C9H10O3 | —H | 165.0557 | 1 | 15.61 | 2 |
| Phosphocreatine | C4H10N3O5P | —H | 210.0285 | 1 | 12.52 | 2 |
| Phosphoenolpyruvate | C3H5O6P | —H | 166.9751 | 1 | 14.29 | 2 |
| Phosphoserine | C3H8NO6P | —H | 184.0016 | 1 | 8.79 | 2 |
| Hydroxyphenyllactic acid | C9H10O4 | —H | 181.0506 | 1 | 12.06 | 2 |
| Proline | C5H9NO2 | —H | 114.0561 | 1 | 1.22 | 2 |
| Pyridoxal | C8H9NO3 | —H | 166.051 | 1 | 2.06 | 3 |
| Pyridoxal 5′-phosphate | C8H10NO6P | —H | 246.0173 | 1 | 13.95 | 2 |
| Pyridoxine | C8H11NO3 | —H | 168.0666 | 1 | 2.13 | 3 |
| Pyroglutamic acid | C5H7NO3 | —H | 128.0353 | 1 | 7.065 | 2 |
| Pyruvate | C3H4O3 | —H | 87.0088 | 1 | 8.64 | 2 |
| Riboflavin | C17H20N4O6 | —H | 375.131 | 1 | 11.9 | 2 |
| SAH | C14H20N6O5S | —H | 383.1143 | 1 | 4.55 | 2 |
| Sedoheptulose 7-phosphate | C7H15O10P | —H | 289.033 | 1 | 7.045 | 2 |
| Serine | C3H7NO3 | —H | 104.0353 | 1 | 1.14 | 2 |
| S-Lactoylglutathione | C13H21N3O8S | —H | 378.0977 | 1 | 13.616 | 2 |
| Succinate | C4H6O4 | —H | 117.0193 | 1 | 12.05 | 2 |
| Taurine | C2H7NO3S | —H | 124.0074 | 1 | 1.18 | 2 |
| Threonic acid | C4H8O5 | —H | 135.0299 | 1 | 5.01 | 2 |
| Threonine | C4H9NO3 | —H | 118.051 | 1 | 1.17 | 2 |
| Thymidine | C10H14N2O5 | —H | 241.083 | 1 | 4.4 | 2 |
| Tryptophan | C11H12N2O2 | —H | 203.0826 | 1 | 7.422 | 2 |
| Tyrosine | C9H11NO3 | —H | 180.0666 | 1 | 1.95 | 3 |
| UDP | C9H14N2O12P2 | —H | 402.9949 | 1 | 13.82 | 2 |
| UDP-alpha-D-glucuronic acid | C15H22N2O18P2 | —H | 579.027 | 1 | 15.88 | 2 |
| UDP-hexose | C15H25N2O17P2 | —H | 566.0556 | 1 | 13.29 | 2 |
| UDP-N-acetylhexosamine | C17H27N3O17P2 | —H | 606.0743 | 1 | 13.37 | 2 |
| UMP | C9H13N2O9P | —H | 323.0286 | 1 | 9.65 | 2 |
| Uracil | C4H4N2O2 | —H | 111.02 | 1 | 1.49 | 2 |
| Ureidosuccinic acid | C5H8N2O5 | —H | 175.036 | 1 | 12.58 | 2 |
| Uric acid | C5H4N4O3 | —H | 167.0211 | 1 | 5.07 | 2 |
| Uridine | C9H12N2O6 | —H | 243.0623 | 1 | 1.95 | 3 |
| UTP | C9H15N2O15P3 | —H | 482.9613 | 1 | 16.09 | 2 |
| Valine | C5H11NO2 | —H | 116.0717 | 1 | 1.35 | 2 |
| Xanthine | C5H4N4O2 | —H | 151.0261 | 1 | 1.7 | 2 |
| Xanthosine | C10H12N4O6 | —H | 283.0684 | 1 | 7.28 | 2 |

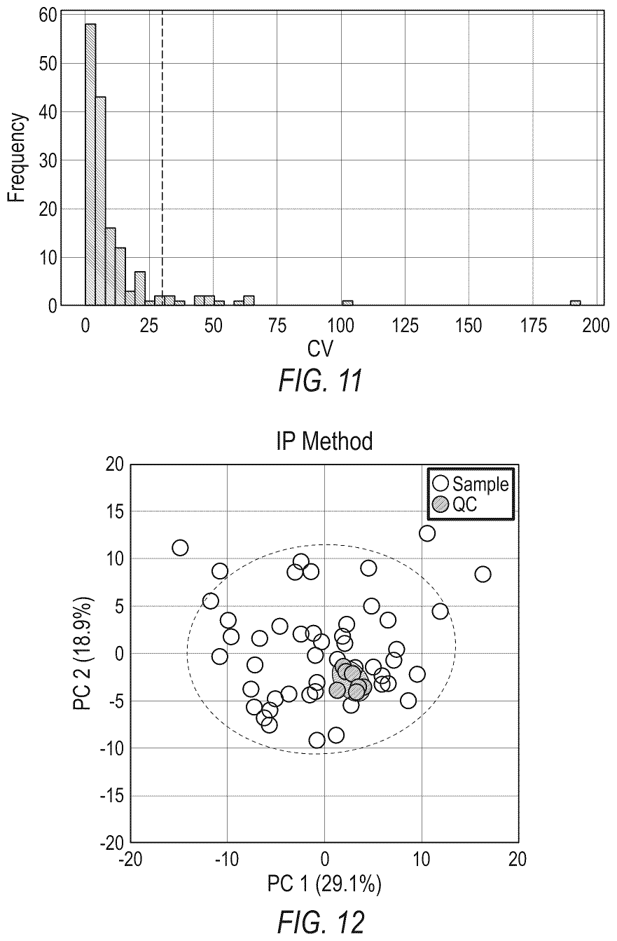

Metabolite peak picking was performed using Skyline software. The exported peak area data was subjected to subsequent data processing steps. Process blank technical controls and pooled quality control (QC) technical controls were used to reduce contributions from noise/background. Process blank technical controls (n=3) consist of solvent that has been subjected to the entire extraction procedure. Pooled QC technical controls (n=7) consist of a mixture of aliquots obtained for all samples, combined at equal volume. Residual unlabeled fatty acid (FA) background levels are commonly observed in the process blank controls due to plastic leaching during the sample preparation procedures. To remove background contributions from the FA analytes in our study (FA 16:0, FA 16:1, FA 18:0, FA 18:1, and FA 20:1), the average abundance/pool size determined across process blanks was subtracted from the level determined in the experimental samples. This procedure was performed at the mass isotopologue distribution (MID) level to ensure isotopologue-specific background levels were subtracted appropriately. In the event this value was negative (i.e., cases where the process blank abundance exceeded the experimental sample level), those values were set to zero. The cellular metabolomics dataset was processed further using a coefficient of variation (CV) filter, performed on the pooled QC technical controls (FIG. 11). Analytes with a CV greater than or equal to 30.0% were excluded from the analysis. Table 4 presents a summary of this procedure.

TABLE 4
Compounds excluded from the metabolomics dataset based on
a coefficient of variation (CV) filter.

| | | | | | Exclusion |
|---|---|---|---|---|---|
| Analyte | μ | s.d. | CV | n^{†} | criteria |
| Adenine | 1071898.5 | 504137.3 | 47.03 | 7 | Exceeded |
| Inosine | 204558.0 | 68305.7 | 33.39 | 7 | CV |
| 3-Hydroxy- | 80474130.0 | 155717076.0 | 193.49 | 7 | threshold |
| isobutyrate | | | | | (>= 30.0%) |
| Glucose-1P | 2750648.4 | 1808229.2 | 65.73 | 7 | |
| 4-Hydroxy- | 6477885.5 | 3453047.6 | 53.30 | 7 | |
| butyrate | | | | | |
| CMP | 191275.7 | 63271.2 | 33.07 | 7 | |
| 2-Hydroxy- | 22775770.7 | 9722297.1 | 42.68 | 7 | |
| butyrate | | | | | |
| S-Lactoyl- | 20280.0 | 9080.1 | 44.77 | 7 | |
| glutathione | | | | | |
| NADPH | 8225.2 | 4796.2 | 58.31 | 7 | |
| N-Acetyl- | 3802.5 | 3854.1 | 101.35 | 7 | |
| methionine | | | | | |
| Coenzyme A | 7462277.2 | 2599487.0 | 34.83 | 7 | |
| Farnesy1-PP | 164982.8 | 102174.5 | 61.93 | 7 | |
| Cytosine | 18721.7 | 8905.9 | 47.57 | 7 | |

^{†}Denotes the number of reference group technical replicates present in the CV analysis.
^{††}The pooled QC technical control consists of a mixture of equal volume aliquots of all samples used in the study.

MID distributions were adjusted to account for contributions from naturally abundant isotopes using the IsoCor python library. Natural abundance correction (NAC) was performed using the parameters provided by Table 5.

TABLE 5
IsoCor natural abundance correction parameters used for
metabolomics data processing.

| Parameter | Value | Notes |
|---|---|---|
| Isotope type | 13 C | |
| Isotope natural abundance | 0.011 | 13 C is 1.1% or 0.011. |
| Tracer purity | 0.0, 1.0 | 100% purity is assumed |
| | | (i.e., 0% 12 C and 100% 13 C) |
| MS resolution | 240000.0 | |
| m/z of resolution | 200.0 | |
| Charge | 1 | |

†Parameter values used when running the python-based IsoCor NAC procedure.

The output of this procedure is the MID pool size dataset used for subsequent analyses. The total pool size table was computed as the sum of the pool size determined for all isotopologues of a given metabolite via Eq. 2, where M=mass and n=the number of carbons:

$$Total Pool Size = {\sum }_{n}^{0} M + 0 , M + 1 , … , M + n \qquad Eq . 2$$

The total labeled pool size was determined by excluding contributions from the M+0 isotopologue, as provided by Eq. 3:

$$Total Labeled Pool Size = {\sum }_{n}^{1} M + 1 , … , M + n \qquad Eq . 3$$

The MID fractional enrichment (MID FE) was determined as the percentage of the pool size accounted for by the isotopologue M+n of a given metabolite, as described by Eq. 4:

$$MID F E = \frac{M + n}{{\sum }_{n}^{0} M + 0 , M + 1 , … , M + n} * 100 \qquad Eq . 4$$

The total label fractional enrichment (total label FE) was determined as the percentage of the pool size that is labeled, as provided by Eq. 5:

$$Total Label F E = \frac{{\sum }_{n}^{1} M + 1 , … , M + n}{{\sum }_{n}^{0} M + 0 , M + 1 , … , M + n} * 100 \qquad Eq . 5$$

Data quality was assessed further by comparing the variation observed in the pooled QC injections relative to the experimental samples. The pooled QC group clustered much more tightly than the experimental samples (FIG. 12). Low variation in pooled QC confirmed measurement consistency across all acquired data.

Lipidomics. Complex lipids were extracted using a modified Bligh-Dyer58 procedure that was previously reported. Briefly, ice cold methanol was added to frozen cells followed by one volume of chloroform (Sigma, 1024441000), resulting in a 1:1 MeOH:chloroform ratio. Samples were vortexed for see and were incubated on ice for 30 min. Afterwards, 0.9 volume of water was added to yield a final ratio of 2:2:1.8 (v/v/v). Phase separation was achieved by vortexing the samples and subjecting them to centrifugation at maximum speed. The organic layer and aqueous layer were collected separately and were dried down via rotary evaporation in a speedvac.

For lipidomics data acquisition, samples were analyzed with a Vanquish liquid chromatography system coupled to an Orbitrap ID-X (Thermo Fisher Scientific) using an H-ESI (heated electrospray ionization) source operating in positive mode. A 2 μL volume of each sample was injected and run through a reversed-phase chromatography Accucore C30 column (2.6 μm, 2.1 mm×150 mm; Thermo Fisher Scientific, 27826-152130) combined with an Accucore C30 guard column (2.6 μm, 2.1 mm×10 mm; Thermo Fisher Scientific, 27826-012105) over a 30 min period. Mobile phase A consisted of 60% LC/MS grade acetonitrile (Fisher Scientific, A955), 40% LC/MS grade water (Fisher, W6), 0.1% LC/MS grade formic acid (Fisher Scientific, A117), 10 mM ammonium formate (Fisher Scientific, 70221), and mobile phase B consisted of 90% LC/MS grade isopropanol (Fisher Scientific, A461), 8% LC/MS grade acetonitrile, 2% LC/MS grade water, 0.1% LC/MS grade formic acid, and 10 mM ammonium formate. Column temperature was kept at 50° C., flow rate was held at 0.4 mL/min, and the chromatography gradient was as follows: 0-1 min held at 25% B, 1-3 min from 25% B to 40% B, 3-19 min from 40% B to 75% B, 19-20.5 min 75% B to 90% B, 20.5-28 min from 90% B to 95% B, 28-28.1 min from 95% B to 100% B, and 28.1-30 min held at 100% B. A 15 minute wash gradient was run between every injection to flush the column and to re-equilibrate solvent conditions as follows: 0-4 min held at 100% B, 4-5 min ramp from 100% B to 25% B, and 5-15 min held at 25% B. Mass spectrometer parameters were: source voltage 3250 V, sheath gas 40, aux gas 10, sweep gas 1, ion transfer tube temperature 300° C., and vaporizer temperature 275° C. Full scan data were collected using the orbitrap with a scan range of 200-1700 m/z at a resolution of 500,000 and RF lens at 45%. Data dependent MS2 fragmentation was induced in the orbitrap using assisted higher-energy collisional dissociation (HCD) collision energies at 15, 30, 45, 75, and 110% as well as with collision-induced dissociation (CID) at a collision energy of 35%. For both MS2 fragmentations, orbitrap resolution was 15,000 and the isolation window was 1.5 m/z. A m/z 184 mass trigger, indicative of phosphatidylcholines, was used for CID fragmentation. Data dependent MS3 fragmentation was induced in the ion trap with scan rate set at Rapid using CID at a collision energy of 35%. MS3 scans were triggered by specific acyl chain losses for detailed analysis of mono-, di-, and triacylglycerides. Total cycle time was 2 sec. Lipid identifications were assigned using LipidSearch (v5.0, Thermo Fisher Scientific).

Lipid peak picking was performed using Skyline software and the exported data were subjected to additional processing steps. Noise filtering was performed using process blank technical controls (n=3) and pooled unlabeled quality controls (QC-NL). QC-NL controls consist of a mixture of equal volume aliquots of all unlabeled samples used in the study. Lipids were excluded if the peak area was less than or equal to a value that was three times the average level observed in process blank controls (Table 6).

TABLE 6
Compounds excluded from the lipidomics dataset due to being
indistinguishable from background levels.

| Analyte | n^{†} | Exclusion criteria | Reference group^{††} |
|---|---|---|---|
| TG(12:0-16:0-16:1) | 3 | | Process blank |
| TG(12:0-16:0-16:0) | 3 | | |
| TG(O-16:0-14:0-16:1) | 3 | | |
| TG(14:0-15:0-16:0) | 3 | | |
| TG(15:1-16:0-16:0) | 3 | | |
| TG(15:0-16:0-16:1) | 3 | | |
| TG(15:0-16:0-16:0) | 3 | | |
| TG(14:0-16:0-17:0) | 3 | | |
| TG(16:0-16:1-17:1) | 3 | | |
| TG(O-18:0-16:0-17:0) | 3 | | |
| TG(O-17:0-16:0-18:0) | 3 | | |
| TG(16:0-18:2-18:2) | 3 | | |
| TG(O-19:0-16:0-18:1) | 3 | | |
| TG(16:0-18:1-18:2) | 3 | | |
| TG(O-16:0-16:0-24:0) | 3 | | |
| TG(16:0-22:0-18:1) | 3 | | |
| TG(24:1-16:0-18:0) | 3 | | |
| TG(16:0-16:0-26:1) | 3 | | |
| TG(16:0-24:0-18:1) | 3 | | |
| TG(16:0-16:0-26:0) | 3 | | |
| TG(16:0-18:1-26:1) | 3 | | |
| TG(50:2-16:0) | 3 | | |
| TG(10:0-16:0-16:0) | 3 | | |
| TG(16:0-26:0-18:1) | 3 | | |
| TG(10:0-14:0-16:0) | 3 | | |
| PC(O-16:0) | 3 | | |
| DG(16:0-24:1) | 3 | | |
| DG(16:0-24:0) | 3 | | |
| DG(16:0-16:1) | 3 | Sample level(s) | |
| DG(16:0-20:0) | 3 | ≤3.0*background | |

^{†}Denotes the number of reference group technical replicates used to compute the average background level.
^{††}The process blank technical control consists of solvent that has been subjected to the entire extraction procedure.

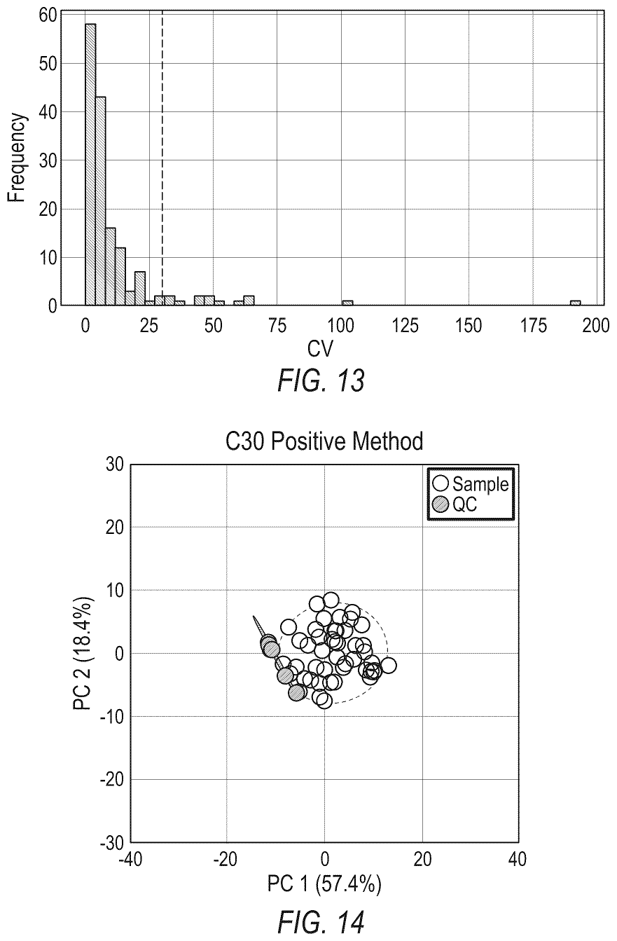

A CV filter was then applied to remove lipids having a CV that was greater than or equal to 30.0% (FIG. 13), determined using QC-NL controls (Table 7).

TABLE 7
Compounds excluded from the lipidomics dataset based on a coefficient of
variation (CV) filter.

| | | | | | Exclusion | Reference |
|---|---|---|---|---|---|---|
| Analyte | μ | s.d. | CV | n^{†} | criteria | group^{††} |
| TG(12:0-16:0-16:1) | 13872022.0 | 11787965.3 | 84.9 | 8 | Exceeded | QC-NL |
| TG(16:0-18:2-18:2) | 2835385394.0 | 1565850935.0 | 55.2 | 8 | CV threshold | |
| TG(16:0-18:1-18:2) | 4901650416.0 | 2416033950.0 | 49.2 | 8 | (>= 30.0%) | |
| PI(20:4-16:0) | 10708464.9 | 3512947.2 | 32.8 | 8 | | |
| PI(22:3-16:0) | 1923759.8 | 801270.5 | 41.6 | 8 | | |
| PI(16:0-16:0) | 13878764.3 | 4219953.7 | 30.4 | 8 | | |
| PE(P-16:0-21:3) | 7963232.3 | 2744886.9 | 34.4 | 8 | | |
| PC(O-16:0-18:1) | 13049832.4 | 8677294.6 | 66.4 | 8 | | |
| PC(16:0-18:0) | 883088447.0 | 337929796.5 | 38.2 | 8 | | |
| PC(O-14:1-16:0) | 79035052.3 | 25163698.1 | 31.8 | 8 | | |

^{†}Denotes the number of reference group technical replicates present in the CV analysis.
^{††}The QC-NL (QC not labeled) technical control consists of a mixture of equal volume aliquots of all unlabeled samples used in the study.

NAC was performed to adjust MID distributions to account for contributions from naturally abundant isotopes using the IsoCor python library. Table 8 describes the parameters used to perform NAC on the lipidomics dataset, which yielded the MID pool size dataset. The total pool size, total labeled pool size, MID FE, and total label FE calculations were performed as described for the metabolomics analysis. Tight clustering was observed for the pooled QC group compared to the experimental samples (FIG. 14), confirming measurement consistency throughout data acquisition.

TABLE 8
IsoCor natural abundance correction parameters used for
lipidomics data processing.

| Parameter | Value | Notes |
|---|---|---|
| Isotope type | 13 C | |
| Isotope natural | 0.011 | 13 C is 1.1% or 0.011. |
| abundance | | |
| Tracer purity | 0.0, 1.0 | 100% purity is assumed (i.e., |
| | | 0% 12 C and 100% 13 C) |
| MS resolution | 500000.0 | |
| m/z of resolution | 200.0 | |
| Charge | 1 | |

^{†}Parameter values used when running the python-based IsoCor NAC procedure.

The present invention has been described in terms of one or more preferred embodiments. It should be appreciated that many equivalents, alternatives, variations, and modifications, aside from those expressly stated, are possible and are within the scope of the invention.

What is claimed is:

1. A composite nanoparticle comprising:
a biodegradable amphiphilic material; and
a hydrophobic compound, wherein the hydrophobic compound includes a stable isotope label.

2. The composite nanoparticle of claim 1, wherein the biodegradable amphiphilic material is a polymeric, non-polymeric, or hybrid polymer/lipid self-assembling system.

3. The composite nanoparticle of claim 1, wherein the biodegradable amphiphilic material is polymeric and is selected from the group consisting of:
a poly(ethylene glycol)-block-poly(ε-caprolactone) copolymer (PEG-b-PCL),
a poly(ethylene glycol)-block-poly(lactide) copolymer (PEG-b-PLA),
a poly(ethylene glycol)-block-poly(lactide-co-glycolide) copolymer (PEG-b-PLGA), and combinations thereof.

4. The composite nanoparticle of claim 1, wherein the biodegradable amphiphilic material is self-assembled.

5. The composite nanoparticle of claim 1, wherein the biodegradable amphiphilic material is a polymer blend.

6. The composite nanoparticle of claim 1, wherein the biodegradable amphiphilic material is a tri-block copolymeric biodegradable amphiphilic material.

7. The composite nanoparticle of claim 6, wherein the tri-block copolymer is a poly(ethylene glycol)-block-poly(lactide)-block-poly(ethylene glycol) copolymer (PEG-b-PLA-b-PEG).

8. The composite nanoparticle of claim 1, wherein the biodegradable amphiphilic material is polymeric and is selected from the group consisting of: a poly(ethylene glycol)-block-poly(carbonate) copolymer, a poly(ethylene glycol)-block-poly(ester) copolymer, a poly(ethylene glycol)-block-poly(amino acid) copolymer, a poly(ethylene glycol)-block-poly(beta-amino ester) copolymer, a poly(ethylene glycol)-block-poly(ortho ester) copolymer, and combinations thereof.

9. The composite nanoparticle of claim 1, wherein the biodegradable amphiphilic material is formed into a micelle, liposome, lipid nanoparticle (LNP), or polymersome.

10. The composite nanoparticle of claim 1, wherein the biodegradable amphiphilic material is a non-polymeric or a hybrid polymer/lipid self-assembling system.

11. The composite nanoparticle of claim 10, wherein the biodegradable amphiphilic material is non-polymeric and is selected from the group consisting of: a phospholipid, an ionizable lipid, a cationic lipid, a neutral lipid, cholesterol, and combinations thereof.

12. The composite nanoparticle of claim 1, wherein the biodegradable amphiphilic material includes a hydrophilic segment and a hydrophobic segment.

13. The composite nanoparticle of claim 12, wherein the hydrophilic segment is poly(ethylene glycol).

14. The composite nanoparticle of claim 13,
wherein the poly(ethylene glycol) is terminated with a moiety selected from the group consisting of a hydroxyl group (—OH), a methyl group (—CH₃), a phosphate group (—PO₄), an amine group (—NH₂), a carboxyl group (—CO₂H), a thiol group (—SH), an aldehyde group (—CHO), a vinyl group (—C₂H₃), an epoxide group, a malemide group, and an azide group (N₃).

15. The composite nanoparticle of claim 2, wherein the poly(ethylene glycol) is methoxy poly(ethylene glycol).

16. The composite nanoparticle of claim 12, wherein the hydrophobic segment is selected from the group consisting of poly(ε-caprolactone), poly(lactide), and poly(lactide-co-glycolide).

17. The composite nanoparticle of claim 16, wherein hydrophobic segment is poly(lactide) in the form of poly(L-lactide), poly(D-lactide), poly(D,L-lactide), or combinations thereof.

18. The composite nanoparticle of claim 16, wherein the hydrophobic segment is poly(lactide-co-glycolide) and the lactide component is in the form of poly(L-lactide), poly(D-lactide), poly(D,L-lactide), or combinations thereof.

19. The composite nanoparticle of claim 12, wherein the hydrophobic segment is selected from the group consisting of: an aliphatic polyester, a polycarbonate, a polyether, a polyurethane, and a lipid tail.

20. The composite nanoparticle of claim 1, wherein the hydrophobic compound is selected from the group consisting of a fatty acid, a fatty acid derivative, and a lipid analog.

21. The composite nanoparticle of claim 1, wherein the hydrophobic compound is selected from the group consisting of palmitate, palmitic acid, palmitoleate, oleate, oleic acid, stearate, linoleate, linolenate, arachidonate, glycerol, triolein, tripalmitin, triglycerides, cholesterol, acetate, choline, ethanolamine, mevalonate, isoprenoids, ceramides, sphingolipids, and a metabolite.

22. The composite nanoparticle of claim 21, wherein the hydrophobic compound is palmitate or oleic acid.

23. The composite nanoparticle of claim 22, wherein the hydrophobic compound is palmitate.

24. The composite nanoparticle of claim 23, wherein the biodegradable amphiphilic material is PEG-b-PCL.

25. The composite nanoparticle of claim 23, wherein the biodegradable amphiphilic material is PEG-b-PLA.

26. The composite nanoparticle of claim 23, wherein the biodegradable amphiphilic material is PEG-b-PLGA.

27. The composite nanoparticle of claim 1, wherein the stable isotope label of the hydrophobic compound is selected from the group consisting of carbon-13 (¹³C), hydrogen-2 (²H), nitrogen-15 (¹⁵N), and oxygen-18 (¹⁸O).

28. The composite nanoparticle of claim 23, wherein the stable isotope label of the hydrophobic compound is ¹³C.

29. The composite nanoparticle of claim 28, wherein the hydrophobic compound is U-¹³C-palmitate.

30. The composite nanoparticle of claim 1, further comprising a hydrophobic fluorescent dye.

31. The composite nanoparticle of claim 1, wherein the composite nanoparticle has an average diameter in a range from about 20 nm to about 400 nm.

32. The composite nanoparticle of claim 31, wherein the average diameter is in the range from about 20 nm to about 50 nm.

33. The composite nanoparticle of claim 1, wherein composite nanoparticle has a surface zeta potential in a range from about −50 mV to about +50 mV.

34. The composite nanoparticle of claim 33, wherein the surface zeta potential is in the range from about −20 mV to about −10 mV.

35. A composition, comprising:
a plurality of composite nanoparticles,
wherein each composite nanoparticle of the plurality of composite nanoparticles includes a biodegradable amphiphilic material and a hydrophobic compound, and
wherein the hydrophobic compound includes a stable isotope label.

36. A pharmaceutical composition, comprising:
the composition of claim 35; and
a pharmaceutically acceptable carrier.

37. A method of following a hydrophobic compound through a biochemical pathway in a cell, comprising the steps of:
providing the cell;
providing a composite nanoparticle, wherein the composite nanoparticle includes a biodegradable amphiphilic material and a hydrophobic compound, and wherein the hydrophobic compound includes a stable isotope label;
delivering an effective amount of the composite nanoparticle to the cell; and
following the hydrophobic compound through the biochemical pathway in the cell.

38. The method of claim 37, wherein the biochemical pathway is selected from the group consisting of central carbon metabolism, lipid biosynthesis, lipid remodeling, lipid transport, and any associated pathway involving the hydrophobic compound or products formed through the participation of the hydrophobic compound in one or more biological reactions.

39. A method of tracking internalization of a hydrophobic compound in a cell, comprising the steps of:
providing the cell;
providing a composite nanoparticle, wherein the composite nanoparticle includes a biodegradable amphiphilic material and a hydrophobic compound, and wherein the hydrophobic compound includes a stable isotope label;
delivering an effective amount of the composite nanoparticle to the cell; and
tracking internalization of the hydrophobic compound by the cell.

40. A method of tracking distribution of a hydrophobic compound in a biological organ or tissue, comprising the steps of:
providing the biological organ or tissue;
providing a composite nanoparticle; wherein the composite nanoparticle includes a biodegradable amphiphilic material and a hydrophobic compound, and wherein the hydrophobic compound includes a stable isotope label
delivering an effective amount of the composite nanoparticle to the biological organ or tissue; and
tracking distribution of the hydrophobic compound in the biological organ or tissue.

41. A method of diagnosing a condition or a disease in a subject, comprising the steps of:
providing the composition of claim 35;
administering a diagnostically effective amount of the composition to the subject, or a cell or tissue of the subject; and
tracking the movement, incorporation, or metabolic effect of the hydrophobic compound in the subject, cell, or tissue.

42. A method of making a composite nanoparticle, wherein the composite nanoparticle includes a biodegradable amphiphilic material and a hydrophobic compound, and wherein the hydrophobic compound includes a stable isotope label, the method comprising the steps of:
providing the biodegradable material copolymer and the hydrophobic compound, and loading the hydrophobic compound into the biodegradable amphiphilic material during or after the self-assembly of the biodegradable amphiphilic material or
providing the biodegradable amphiphilic material and the hydrophobic compound, and loading the hydrophobic compound into the biodegradable amphiphilic material after the self-assembly of the biodegradable amphiphilic material.
